(19) United States
(12) Patent Application Publication
(10) Pub. No.: US 2002/0151595 A1
(43) Pub. Date: Oct. 17, 2002

(54) Carboxylic acid amides having antithrombotic activity

(76) Inventors: Uwe Joerg Ries, Biberach (DE); Henning Priepke, Warthausen (DE); Herbert Nar, Ochsenhausen (DE); Jean-Marie Stassen, Leuven (BE); Wolfgang Wienen, Biberach/Rissegg (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/051,412

(22) Filed: Jan. 17, 2002

Related U.S. Application Data
(60) Provisional application No. 60/269,043, filed on Feb. 16, 2001.

(30) Foreign Application Priority Data
Feb. 2, 2001 (DE) DE 101 04 598.0
Jul. 26, 2001 (DE) DE 101 36 434.2

(51) Int. Cl. A61K 31/455; A61K 31/401; C07C 237/28

(52) U.S. Cl. 514/617; 514/423; 514/354; 546/314; 548/530; 564/152; 564/182

(57) ABSTRACT

Compounds with antithrombotic activity and factor Xa-inhibiting activity of the general formula:

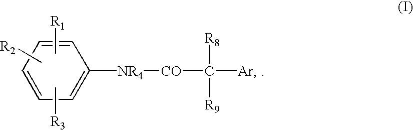

Exemplary are:

(1) (L)-2-(5-carbamimidoyl-2-hydroxy-phenyl)-N-[3-methyl-4-(2-aminocarbonyl-pyrrolidin-1-yl-carbonyl)-phenyl]-acetamide,

(2) 2-(5-carbamimidoyl-2-hydroxy-phenyl)-N-{3-methyl-4-[2-(tert.butoxycarbonyl-aminomethyl)-piperidin-1-yl-carbonyl]-phenyl}-acetamide, and

(3) 2-(5-aminomethyl-2-hydroxy-phenyl)-N-[3-methyl-4-(pyrrolidin-1-yl-carbonyl)-phenyl]-acetamide.

### RELATED APPLICATIONS

Benefit of U.S. Provisional Application Serial No. 60/109,371, filed on Nov. 23, 1998 is hereby claimed.

### DESCRIPTION OF THE INVENTION

The present invention relates to carboxylic acid amides of general formula

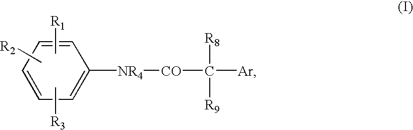

the tautomers, the stereoisomers, the mixtures, the prodrugs, the derivatives thereof which contain a group that is negatively charged under physiological conditions instead of a carboxy group, and the salts thereof, particularly the physiologically acceptable salts thereof with inorganic or organic acids or bases which have valuable properties.

The compounds of the above general formula I wherein Ar denotes a phenyl or naphthyl group substituted by the groups R₅, R₆ and R₇, and R₅ denotes a cyano group, are valuable intermediate products for preparing the corresponding compounds of general formula I wherein R₅ denotes an amidino group optionally substituted by one or two C₁₋₃-alkyl groups. The compounds of the above general formula I with the exception of those compounds wherein Ar denotes a phenyl or naphthyl group substituted by the groups R₅, R₆ and R₇, and R₅ denotes a cyano group, as well as the tautomers, the stereoisomers, the mixtures, the prodrugs, the derivatives thereof which contain a group that is negatively charged under physiological conditions instead of a carboxy group, and the salts thereof, particularly the physiologically acceptable salts thereof with inorganic or organic acids, and the stereoisomers thereof, have valuable pharmacological properties, particularly an antithrombotic activity and an inhibiting effect on factor Xa. The present application thus relates to the new compounds of the above general formula I and the compounds

2-(5-carbamimidoyl-2-hydroxy-phenyl)-N-[3-chloro-4-(pyrrolidin-1-yl-carbonyl)-phenyl]-acetamide,

2-(5-carbamimidoyl-2-hydroxy-phenyl)-N-[3-methyl-4-(pyrrolidin-1-yl-sulphonyl)-phenyl]-acetamide,

2-(5-carbamimidoyl-2-hydroxy-phenyl)-N-[3-bromo-4-(pyrrolidin-1-yl-carbonyl)-phenyl]-acetamide and

2-(5-carbamidoyl-2-hydroxy-phenyl)-N-[3-methoxy-4-(pyrrolidin-1-yl-carbonyl)-phenyl]-acetamide,

the preparation thereof, the pharmaceutical compositions containing the pharmacologically effective compounds, their preparation and use.

In the above general formula

R₁ denotes a C₃₋₇-cycloalkyl-carbonyl group wherein

the methylene group in the 3 or 4 position in a C₅₋₇-cycloalkyl-carbonyl group may be replaced by an —NH group wherein

the hydrogen atom of the —NH group may be replaced by a C₁₋₃-alkyl, C₁₋₃-alkyl-carbonyl, phenylcarbonyl or phenylsulphonyl group,

a C₁₋₆-alkylcarbonyl group optionally terminally substituted in the alkyl moiety by an amino, C₁₋₃-alkylamino or di-(C₁₋₃-alkyl)-amino group,

a group of formula

R_{f}R_{g}N—(CH₂)ₘ—(Rₕ)N—CO

wherein

R_{f}, R_{g} and Rₕ independently of one another each denote a hydrogen atom or a

C₁₋₃-alkyl group and

m denotes one of the numbers 2, 3, 4, 5 or 6,

a phenylcarbonyl, naphthylcarbonyl or heteroarylcarbonyl group,

a C₁₋₃-alkyl group monosubstituted by a hydroxy group or terminally disubstituted by a phenyl and a hydroxy group wherein

the phenyl substituent may be substituted by an amidino group optionally substituted by one or two C₁₋₃-alkyl groups, by a fluorine, chlorine or bromine atom, by a trifluoromethyl, C₁₋₃-alkyl or C₁₋₃-alkoxy group,

a 4- to 7-membered cycloalkyleneimino-carbonyl or cycloalkyleneimino-sulphonyl group substituted by an amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl or di-(C₁₋₃-alkyl)-aminocarbonyl group,

a C₃₋₇-cycloalkylamino group which is substituted at the nitrogen atom by a C₁₋₃-alkyl-amino-C₁₋₃-alkyl or di-(C₁₋₃-alkyl)amino-C₁₋₃-alkyl group,

or, if R₂ denotes a trifluoromethyl group and/or R₅ denotes an amino-C₁₋₃-alkyl, C₁₋₃-alkylamino-C₁₋₃-alkyl or di-(C₁₋₃-alkyl)amino-C₁₋₃-alkyl group and/or R₆ denotes a carboxy-C₁₋₃-alkoxy or C₁₋₄-alkoxy-carbonyl-C₁₋₃-alkoxy group and/or at least one of the groups R₈ or R₉ assumes a meaning other than the hydrogen atom, an unsubstituted 4- to 7-membered cycloalkyleneimino-carbonyl or cycloalkyleneimino-sulphonyl group, a C₃₋₇-cycloalkylamino or N-(C₁₋₃-alkyl)-C₃₋₇-cycloalkylamino group,

R₂ denotes a hydrogen, fluorine, chlorine or bromine atom, a C₁₋₃-alkyl group wherein the hydrogen atoms may be wholly or partly replaced by fluorine atoms, a hydroxy or C₁₋₃-alkoxy group,

R₃ denotes a hydrogen atom or a C₁₋₃-alkyl group,

R₄ denotes a hydrogen atom or a C₁₋₃-alkyl group optionally substituted by a carboxy group or a group which may be converted into a carboxy group in vivo,

Ar denotes a phenyl or naphthyl group substituted by the groups R₅, R₆ and R₇, while

R₅ denotes a cyano group, an amidino group optionally substituted by one or two C₁₋₃-alkyl groups, an amino-C₁₋₃-alkyl, C₁₋₃-alkylamino-C₁₋₃-alkyl or di-(C₁₋₃-alkyl)amino-C₁₋₃-alkyl group,

R₆ denotes a hydrogen, fluorine, chlorine or bromine atom, a trifluoromethyl, C₁₋₃-alkyl, hydroxy, hydroxy-C₁₋₃-alkyl, C₁₋₃-alkoxy, C₁₋₃-alkoxy-C₁₋₃-alkyl, carboxy, carboxy-C₁₋₃-alkyl, carboxy-C₁₋₃-alkoxy, C₁₋₄-alkoxy-carbonyl-C₁₋₃-alkoxy, phenyl-C₁₋₃-alkoxy, amino, C₁₋₃-alkylamino or di-(C₁₋₃-alkyl)amino group and

R₇ denotes a hydrogen, fluorine, chlorine or bromine atom or a C₁₋₃-alkyl group,

or a thienylene, thiazolylene, pyridinylene, pyrimidinylene, pyrazinylene or pyridazinylene group optionally substituted in the carbon skeleton by a C₁₋₃-alkyl group,

R₈ and R₉, which may be identical or different, each denote a hydrogen atom, a C₁₋₃-alkyl group optionally substituted by a phenyl or heteroaryl group or an amino group optionally substituted by one or two C₁₋₃-alkyl or C₁₋₃-alkyl-carbonyl groups,

while the term heteroaryl group mentioned above denotes a 5-membered heteroaryl group bound via a carbon or nitrogen atom which contains

an imino group optionally substituted by a C₁₋₄-alkyl or C₁₋₄-alkyl-carbonyl group, an oxygen or sulphur atom,

an imino group optionally substituted by a C₁₋₄-alkyl group or an oxygen or sulphur atom and additionally a nitrogen atom,

an imino group optionally substituted by a C₁₋₄-alkyl group and two nitrogen atoms or

an oxygen or sulphur atom and two nitrogen atoms,

or a 6-membered heteroaryl group which contains one or two nitrogen atoms,

while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety,

and the unsubstituted or monosubstituted phenyl and naphthyl groups mentioned in the definition of the abovementioned groups, or the unsubstituted or monosubstituted phenyl and naphthyl groups contained in these groups, as well as the abovementioned heteroaryl groups may additionally be substituted at a carbon atom in each case by a fluorine, chlorine or bromine atom, by a trifluoromethyl, C₁₋₃-alkyl, C₁₋₃-alkoxy or C₁₋₃-alkoxy-carbonyl group, unless otherwise stated.

The carboxy groups mentioned in the definition of the abovementioned groups may be replaced by a group which may be converted in vivo into a carboxy group or by a group which is negatively charged under physiological conditions,

and moreover the amino and imino groups mentioned in the definition of the abovementioned groups may be substituted by a group which can be cleaved in vivo. Such groups are described for example in WO 98/46576 and by N. M. Nielsen et al. in International Journal of Pharmaceutics 39, 75-85 (1987).

By a group which can be converted in vivo into a carboxy group is meant, for example, a hydroxymethyl group, a carboxy group esterified with an alcohol wherein the alcohol moiety is preferably a C₁₋₆-alkanol, a phenyl-C₁₋₃-alkanol, a C₃₋₉-cycloalkanol, while a C₅₋₈-cycloalkanol may additionally be substituted by one or two C₁₋₃-alkyl groups, a C₅₋₈-cycloalkanol wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxycarbonyl or C₂₋₆-alkanoyl group and the cycloalkanol moiety may additionally be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol with the that no bonds to the oxygen atom start from a carbon atom which carries a double or triple bond, a C₃₋₈-cycloalkyl-C₁₋₃-alkanol, a bicycloalkanol with a total of 8 to 10 carbon atoms which may additionally be substituted in the bicycloalkyl moiety by one or two C₁₋₃-alkyl groups, a 1,3-dihydro-3-oxo-1-isobenzofuranol or an alcohol of formula

Rₐ—CO—O—(R_{b}CR_{c})—OH,

wherein

Rₐ denotes a C₁₋₈-alkyl, C₅₋₇-cycloalkyl, phenyl or phenyl-C₁₋₃-alkyl group,

R_{b} denotes a hydrogen atom, a C₁₋₃-alkyl, C₅₋₇-cycloalkyl or phenyl group and

R_{c} denotes a hydrogen atom or a C₁₋₃-alkyl group,

by a group which is negatively charged under physiological conditions is meant, for example, a tetrazol-5-yl, phenylcarbonylaminocarbonyl,

trifluoromethylcarbonylaminocarbonyl, C₁₋₆-alkylsulphonylamino, phenylsulphonylamino, benzylsulphonylamino, trifluoromethylsulphonylamino,

C₁₋₆-alkylsulphonylaminocarbonyl, phenylsulphonylaminocarbonyl, benzylsulphonylaminocarbonyl or perfluoro-C₁₋₆-alkylsulphonylaminocarbonyl group and by a group which can be cleaved in vivo from an imino or amino group is meant, for example, a hydroxy group, an acyl group such as a phenylcarbonyl group optionally mono- or disubstituted by fluorine, chlorine, bromine or iodine atoms, by C₁₋₃-alkyl or C₁₋₃-alkoxy groups, while the substituents may be identical or different, a pyridinoyl group or a C₁₋₁₆-alkanoyl group such as the formyl, acetyl, propionyl, butanoyl, pentanoyl or hexanoyl group, a 3,3,3-trichloropropionyl or allyloxycarbonyl group, a C₁₋₁₆-alkoxycarbonyl or C₁₋₁₆-alkylcarbonyloxy group, wherein hydrogen atoms may be wholly or partially replaced by fluorine or chlorine atoms such as the methoxycarbonyl, ethoxycarbonyl, propoxycarbonyl, isopropoxycarbonyl, butoxycarbonyl, tert.butoxycarbonyl, pentoxycarbonyl, hexyloxycarbonyl, octyloxycarbonyl, nonyloxycarbonyl, decyloxycarbonyl, undecyloxycarbonyl, dodecyloxycarbonyl, hexadecyloxycarbonyl, methylcarbonyloxy, ethylcarbonyloxy, 2,2,2-trichloroethylcarbonyloxy, propylcarbonyloxy, isopropylcarbonyloxy, butylcarbonyloxy, tert.butylcarbonyloxy, pentylcarbonyloxy, hexylcarbonyloxy, octylcarbonyloxy, nonylcarbonyloxy, decylcarbonyloxy, undecylcarbonyloxy, dodecylcarbonyloxy or hexadecylcarbonyloxy group, a phenyl-C₁₋₆-alkoxycarbonyl group such as the benzyloxycarbonyl, phenylethoxycarbonyl or phenylpropoxycarbonyl group, a 3-amino-propionyl group wherein the amino group may be mono- or disubstituted by C₁₋₆-alkyl or C₃₋₇-cycloalkyl groups and the substituents may be identical or different, a C₁₋₃-alkylsulphonyl-C₂-₄-alkoxycarbonyl, C₁₋₃-alkoxy-C₂₋₄-alkoxy-C₂₋₄-alkoxycarbonyl, Rₐ—CO—O—(R_{b}CR_{c})—O—CO, C₁₋₆-alkyl-CO—NH—(R_{d}CRₑ)—O—CO or C₁₋₆-alkyl-CO—O—(R_{d}CRₑ)—(R_{d}CRₑ)—O—CO— group, wherein Rₐ to R_{c} are as hereinbefore defined,

R_{d} and Rₑ, which may be identical or different, denote hydrogen atoms or C₁₋₃-alkyl groups.

Moreover, the saturated alkyl and alkoxy moieties containing more than 2 carbon atoms mentioned in the definitions above also include the branched isomers thereof such as the isopropyl, tert.butyl, isobutyl group, etc.

Preferred compounds of the above general formula I are those wherein

R₁ denotes a C₅₋₇-cycloalkyl-carbonyl group wherein the methylene group in the 3 or 4 position is replaced by an —NH group wherein

the hydrogen atom may be replaced by a C₁₋₃-alkyl, C₁₋₃-alkyl-carbonyl or phenyl-carbonyl group,

a C₁₋₃-alkyl-carbonyl group optionally terminally substituted in the alkyl moiety by a C₁₋₃-alkylamino or di-(C₁₋₃-alkyl)-amino group,

a group of formula

R_{f}R_{g}N—(CH₂)ₘ—(Rₕ)N—CO,

wherein

R_{f}, R_{g} and Rₕ independently of one another each denote a hydrogen atom or a C₁₋₃-alkyl group and

m denotes one of the numbers 2, 3 or 4,

a phenylcarbonyl or heteroarylcarbonyl group,

while the heteroaryl moiety contains a 6-membered heteroaryl group which contains one or two nitrogen atoms and to which a phenyl ring may be fused via two adjacent carbon atoms, while the bicyclic heteroaryl groups thus formed may be bound via the heteroaromatic or carbocyclic moiety, e.g. a 2-pyridyl, 3-pyridyl, 4-pyridyl, pyrazinyl, pyrimidinyl, pyridazinyl, quinolinyl, isoquinolinyl, quinoxalinyl or quinazolinyl group,

a C₁₋₃-alkyl group monosubstituted by a hydroxy group or terminally disubstituted by a phenyl group and a hydroxy group wherein

the phenyl substituent may be substituted by an amidino group optionally substituted by one or two C₁₋₃-alkyl groups, by a fluorine, chlorine or bromine atom, by a trifluoromethyl, C₁₋₃-alkyl or C₁₋₃-alkoxy group,

a 4- to 7-membered cycloalkyleneimino-carbonyl group substituted by an amino-C₁₋₃-alkyl, C₁₋₃-alkylamino-C₁₋₃-alkyl, di-(C₁₋₃-alkyl)-amino-C₁₋₃-alkyl, C-₄-alkoxy-carbonyl-amino-C₁₋₃-alkyl, aminocarbonyl, C₁₋₃-alkylamino-carbonyl or di-(C₁₋₃-alkyl)-aminocarbonyl group,

a C₅₋₇-cycloalkylamino group which is substituted at the nitrogen atom by a C₁₋₃-alkyl-amino-c₁₋₃-alkyl or di-(C₁₋₃-alkyl)amino-C₁₋₃-alkyl group,

or, if R₂ denotes a trifluoromethyl group and/or R₅ denotes an amino-C₁₋₃-alkyl or C₁₋₃-alkylamino-C₁₋₃-alkyl group and/or R₆ denotes a carboxy-C₁₋₃-alkoxy or C₁₋₄-alkoxy-carbonyl-C₁₋₃-alkoxy group and/or at least one of the groups R₈ or R₉ assumes a meaning other than the hydrogen atom, an unsubstituted 4- to 7-membered cycloalkyleneiminocarbonyl group, a C₅₋₇-cycloalkylamino or N-(C₁₋₃-alkyl)-C₅₋₇-cycloalkylamino group,

R₂ denotes a hydrogen, fluorine, chlorine or bromine atom, a C₁₋₃-alkyl, trifluoromethyl or C₁₋₃-alkoxy group,

R₃ denotes a hydrogen atom or a C₁₋₃-alkyl group,

R₄ denotes a hydrogen atom or a C₁₋₃-alkyl group,

Ar denotes a phenyl group substituted by the groups R₅ and R₆ wherein

R₅ denotes a cyano group, an amidino group optionally substituted by one or two C₁₋₃-alkyl groups, an amino-C₁₋₃-alkyl or C₁₋₃-alkylamino-C₁₋₃-alkyl group and

R₆ denotes a hydrogen, fluorine, chlorine or bromine atom, a trifluoromethyl, C₁₋₃-alkyl, hydroxy, C₁₋₃-alkoxy, carboxy-C₁₋₃-alkoxy or C₁₋₄-alkoxy-carbonyl-C₁₋₃-alkoxy group, and

R₈ and R₉, which may be identical or different, each denote a hydrogen atom, a C₁₋₃-alkyl group optionally substituted by an phenyl or pyridinyl group or an amino group optionally substituted by one or two C₁₋₃-alkyl or C₁₋₃-alkyl-carbonyl groups,

while the unsubstituted or monosubstituted phenyl groups mentioned in the definition of the abovementioned groups, or the unsubstituted or monosubstituted phenyl moieties contained in these groups, as well as the abovementioned heteroaryl groups may additionally be substituted at a carbon atom in each case by a fluorine, chlorine or bromine atom, by a trifluoromethyl, C₁₋₃-alkyl, C₁₋₃-alkoxy or C₁₋₃-alkoxy-carbonyl group, unless otherwise stated,

and the compounds

2-(5-carbamimidoyl-2-hydroxy-phenyl)-N-[3-chloro-4-(pyrrolidin-1-yl-carbonyl)-phenyl]-acetamide,

2-(5-carbamimidoyl-2-hydroxy-phenyl)-N-[3-methyl-4-(pyrrolidin-1-yl-sulphonyl)-phenyl]-acetamide,

2-(5-carbamimidoyl-2-hydroxy-phenyl)-N-[3-bromo-4-(pyrrolidin-1-yl-carbonyl)-phenyl]-acetamide and

2-(5-carbamidoyl-2-hydroxy-phenyl)-N-[3-methoxy-4-(pyrrolidin-1-yl-carbonyl)-phenyl]-acetamide,

the stereoisomers and the salts thereof, but particularly those compounds wherein

the groups R₁ to R₄, R₈ and R₉ are as hereinbefore defined, but R₁ in the 4 position is bound to the phenyl group contained in formula I and

Ar denotes a phenyl group disubstituted by the groups R₅ and R_{6,} while

R₅ is bound in the 3 position if R₆ denotes a hydrogen atom, or is bound in the 5 position if R₆ assumes a meaning other than the hydrogen atom, and an amidino group optionally substituted by one or two C₁₋₃-alkyl groups, an amino-C₁₋₃-alkyl or C₁₋₃-alkylamino-C₁₋₃-alkyl group and

R₆ denotes a hydrogen atom or a trifluoromethyl, C₁₋₃-alkyl, hydroxy, C₁₋₃-alkoxy, carboxy-C₁₋₃-alkoxy or C₁₋₄-alkoxy-carbonyl-C₁₋₃-alkoxy group bound in the 2 position,

and the compounds

2-(5-carbamimidoyl-2-hydroxy-phenyl)-N-[3-chloro-4-(pyrrolidin-1-yl-carbonyl)-phenyl]-acetamide and

2-(5-carbamimidoyl-2-hydroxy-phenyl)-N-[3-bromo-4-(pyrrolidin-1-yl-carbonyl)-phenyl]-acetamide,

the stereoisomers and the salts thereof.

Particularly preferred compounds of general formula I are those wherein

R₁ is bound in the 4 position of the phenyl group of formula I and denotes

a C₅₋₇-cycloalkyl-carbonyl group wherein the methylene group in the 3 or 4 position is replaced by an —NH group,

a phenylcarbonyl or pyridylcarbonyl group optionally substituted by a fluorine, chlorine or bromine atom or by a C₁₋₃-alkyl group,

a C₁₋₃-alkyl group terminally disubstituted by a phenyl and a hydroxy group wherein

the phenyl substituent may be monosubstituted by a C₁₋₃-alkyl or an amidino group or may be disubstituted by a C₁₋₃-alkyl and an amidino group,

a 5- to 7-membered cycloalkyleneimino-carbonyl group substituted by an amino-C₁₋₃-alkyl, C₁₋₃-alkylamino-C₁₋₃-alkyl, C₁₋₄-alkoxy-carbonyl-amino-C₁₋₃-alkyl, aminocarbonyl or C₁₋₃-alkylamino-carbonyl group,

or, if R₂ denotes a trifluoromethyl group and/or R₅ denotes an amino-C₁₋₃-alkyl group and/or R₆ denotes a carboxy-C₁₋₃-alkoxy or C₁₋₄-alkoxy-carbonyl-C₁₋₃-alkoxy group and/or at least one of the groups R₈ or R₉ assumes a meaning other than the hydrogen atom, an unsubstituted 5- to 7-membered cycloalkyleneimino-carbonyl or cycloalkyleneimino-sulphonyl group and

R₂ denotes a hydrogen atom or a substitutent bound in the 3 position of the phenyl group, selected from among fluorine, chlorine, bromine, C₁₋₃-alkyl, C₁₋₃-alkoxy and trifluoromethyl,

R₃ and R₄ each denote a hydrogen atom,

Ar denotes a phenyl group substituted by the groups R₅ and R₆ wherein

R₅ is bound in the 3 position if R₆ denotes a hydrogen atom, or is bound in the 5 position if R₆ assumes a meaning other than the hydrogen atom, and an amidino or amino-C₁₋₃-alkyl group and

R₆ denotes a hydrogen atom or a hydroxy, C₁₋₃-alkoxy, carboxy-C₁₋₃-alkoxy or C₁₋₄-alkoxy-carbonyl-C₁₋₃-alkoxy group bound in the 2 position, and

R₈ and R₉, which may be identical or different, each denote a hydrogen atom, a C₁₋₃-alkyl group optionally substituted by a phenyl, 4-(C₁₋₃-alkoxy-carbonyl)-phenyl or pyridinyl group or an amino group optionally substituted by one or two C₁₋₃-alkyl or C₁₋₃-alkyl-carbonyl groups,

and the compounds

2-(5-carbamimidoyl-2-hydroxy-phenyl)-N-[3-chloro-4-(pyrrolidin-1-yl-carbonyl)-phenyl]-acetamide and

2-(5-carbamimidoyl-2-hydroxy-phenyl)-N-[3-bromo-4-(pyrrolidin-1-yl-carbonyl)-phenyl]-acetamide,

the stereoisomers and the salts thereof

The following preferred compounds are mentioned by way of example:

(1) (L)-2-(5-carbamimidoyl-2-hydroxy-phenyl)-N-[3-methyl-4-(2-aminocarbonyl-pyrrolidin-1-yl-carbonyl)-phenyl]-acetamide,

(2) 2-(5-carbamimidoyl-2-hydroxy-phenyl)-N-{3-methyl-4-[2-(tert.butoxycarbonyl-aminomethyl)-piperidin-1-yl-carbonyl]-phenyl}-acetamide,

(3) 2-(5-aminomethyl-2-hydroxy-phenyl)-N-[3-methyl-4-(pyrrolidin-1-yl-carbonyl)-phenyl]-acetamide,

(4) 2-(3-carbamimidoyl-phenyl)-N-[3-methyl-4-(pyrrolidin-1-yl-carbonyl)-phenyl]-isobutyramide,

(5) 2-(5-carbamimidoyl-2-ethoxycarbonylmethyloxy-phenyl)-N-[3-methyl-4-(pyrrolidin-1-yl-carbonyl)-phenyl]-acetamide,

(6) 2-(5-carbamimidoyl-2-carboxymethyloxy-phenyl)-N-[3-methyl-4-(pyrrolidin-1-yl-carbonyl)-phenyl]-acetamide,

(7) 2-(5-carbamimidoyl-2-hydroxy-phenyl)-N-[3-methyl-4-(piperidin-3-yl-carbonyl)-phenyl]-acetamide,

(8) 2-(5-carbamimidoyl-2-hydroxy-phenyl)-N-(3-methyl-4-benzoyl-phenyl)-acetamide,

(9) 2-(5-carbamimidoyl-2-hydroxy-phenyl)-N-[3-methyl-4-(1-hydroxy-1-phenyl-methyl)-phenyl]-acetamide,

(10) 2-(5-carbamimidoyl-2-hydroxy-phenyl)-N-{4-[1-(3-carbamimidoyl-phenyl)-1-hydroxy-methyl]-3-methyl-phenyl}-acetamide,

(11) 2-(3-carbamidoyl-phenyl)-N-[3-methyl-4-(pyridin-3-yl-carbonyl)-phenyl]-isobutyramide,

(12) 2-(5-carbamimidoyl-2-hydroxy-phenyl)-N-[3-methyl-4-(pyrrolidin-1-yl-carbonyl)-phenyl]-isobutyramide,

(13) 2-(5-carbamimidoyl-2-hydroxy-phenyl)-N-[3-chloro-4-(pyrrolidin-1-yl-carbonyl)-phenyl]-acetamide,

(14) 2-(3-carbamimidoyl-phenyl)-N-[3-methyl-4-(pyrrolidin-1-yl-carbonyl)-phenyl]-2-amino-acetamide,

(15) 2-(3-carbamimidoyl-phenyl)-N-[3-methyl-4-(pyrrolidin-1-yl-carbonyl)-phenyl]-2-(acetylamino)-acetamide,

(16) 2-(5-carbamimidoyl-2-hydroxy-phenyl)-N-[3-methyl-4-(pyrrolidin-1-yl-sulphonyl)-phenyl]-acetamide,

(17) 2-(5-carbamimidoyl-2-hydroxy-phenyl)-N-[3-methyl-4-(pyrrolidin-1-yl-carbonyl)-phenyl]-propionamide,

(18) 2-(5-carbamimidoyl-2-hydroxy-phenyl)-N-[3-trifluoromethyl-4-(pyrrolidin-1-yl-carbonyl)-phenyl]-acetamide,

(19) 2-(5-carbamimidoyl-2-hydroxy-phenyl)-N-[4-(pyrrolidin-1-yl-carbonyl)-3-trifluoromethyl-phenyl]-propionamide,

(20) 2-(5-carbamimidoyl-2-hydroxy-phenyl)-N-[3-bromo-4-(pyrrolidin-1-yl-carbonyl)-phenyl]-acetamide,

(21) 2-(5-carbamimidoyl-2-hydroxy-phenyl)-N-[3-bromo-4-(pyrrolidin-1-yl-carbonyl)-phenyl]-propionamide,

(22) 2-(5-carbamidoyl-2-hydroxy-phenyl)-N-[3-methoxy-4-(pyrrolidin-1-yl-carbonyl)-phenyl]-acetamide,

(23) 2-(3-carbamimidoyl-phenyl)-N-[3-bromo-4-(pyrrolidin-1-yl-carbonyl)-phenyl]-3-phenyl-propionamide,

(24) 2-(3-carbamimidoyl-phenyl)-N-[3-bromo-4-(pyrrolidin-1-yl-carbonyl)-phenyl]-3-(pyridin-4-yl)-propionamide and

(25) 2-(3-carbamimidoyl-phenyl)-N-[3-bromo-4-(pyrrolidin-1-yl-carbonyl)-phenyl]-3-[4-(ethoxycarbonyl)-phenyl]-propionamide,

wherein the amidino group may additionally be substituted by a C₁₋₆-alkoxycarbonyl or phenylcarbonyl group, and the salts thereof.

According to the invention, the compounds of general formula I are obtained by methods known per se, e.g. by the following processes:

a) In order to prepare a compound of general formula I wherein Ar denotes a phenyl or naphthyl group substituted by the groups R₅, R₆ and R₇, while R₆ and R₇ are as hereinbefore defined and R₅ denotes an amidino group:

acylating a compound of general formula

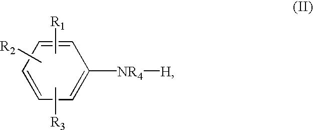

wherein R₁ to R₄ are as hereinbefore defined, with a carboxylic acid of general formula

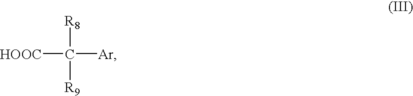

wherein R₈ and R₉ are as hereinbefore defined and Ar denotes a phenyl or naphthyl group substituted by the groups R₅, R₆ and R₇, while R₅ denotes a cyano group and R₆ and R₇ are as hereinbefore defined, or with the reactive derivatives thereof and subsequently converting the cyano compound thus obtained into an amidino compound.

The acylation is conveniently carried out with a corresponding halide or anhydride in a solvent such as methylene chloride, chloroform, carbon tetrachloride, ether, tetrahydrofuran, dioxan, benzene, toluene, acetonitrile, dimethylformamide or sulpholane optionally in the presence of an inorganic or organic base at temperatures between −20 and 200° C., but preferably at temperatures between −10 and 160° C.

The acylation may however also be carried out with the free acid or an ester, optionally in the presence of an acid-activating agent or a dehydrating agent, e.g. in the presence of isobutyl chloroformate, thionyl chloride, trimethylchlorosilane, hydrogen chloride, sulphuric acid, methanesulphonic acid, p-toluenesulphonic acid, phosphorus trichloride, phosphorus pentoxide, triethylamine, N,N′-dicyclohexylcarbodiimide, N,N′-dicyclohexylcarbodiimide/N-hydroxysuccinimide or 1-hydroxy-benzotriazole, O-(benzotriazol-1-yl)-N,N,N′,N′-tetramethyl-uronium tetrafluoroborate/N-methyl-morpholine, propanephosphonic acid-cycloanhydride/N-methylmorpholine, N,N′-carbonyldiimidazole or N,N′-thionyldiimidazole or triphenylphosphine/carbon tetrachloride, at temperatures between −20 and 200° C., but preferably at temperatures between −10 and 160° C.

The subsequent conversion of the cyano group into an amidino group takes place as described in process b).

b) In order to prepare a compound of general formula I wherein Ar denotes a phenyl or naphthyl group substituted by the groups R₅, R₆ and R₇, while R₆ and R₇ are as hereinbefore defined and R₅ denotes an amidino group optionally substituted by one or two C₁₋₃-alkyl groups:

reacting a compound of general formula

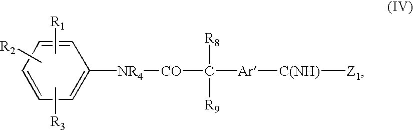

optionally formed in the reaction mixture, wherein

R₁ to R₄, R₈ and R₉ are as hereinbefore defined, Ar′ denotes a phenyl or naphthyl group substituted by the groups R₆ and R₇, while R₆ and R₇ are as hereinbefore defined, and

Z₁ denotes an alkoxy or aralkoxy group such as the methoxy, ethoxy, n-propoxy, iso-propoxy or benzyloxy group or an alkylthio or aralkylthio group such as the methylthio, ethylthio, n-propylthio or benzylthio group, with an amine of general formula

H—R₁₀NR₁₁, (V)

wherein

R₁₀ and R₁₁, which may be identical or different, each denote a hydrogen atom, a C₁₋₃-alkyl or an amino group optionally substituted by one or two C₁₋₃-alkyl or C₁₋₃-alkyl-carbonyl groups, or with the salts thereof.

The reaction is conveniently carried out in a solvent such as methanol, ethanol, n-propanol, tetrahydrofuran or dioxan at temperatures between 0 and 150° C., preferably at temperatures between 0 and 80° C., with an amine of general formula V or with a corresponding acid addition salt such as for example ammonium carbonate or ammonium acetate.

A compound of general formula IV is obtained for example by reacting a corresponding cyano compound with a corresponding alcohol such as methanol, ethanol, n-propanol, isopropanol or benzyl alcohol in the presence of an acid such as hydrochloric acid or by reacting a corresponding amide with a trialkyloxonium salt such as triethyloxonium-tetrafluoroborate in a solvent such as methylene chloride, tetrahydrofuran or dioxan at temperatures between 0 and 50° C., but preferably at 20° C., or a corresponding nitrile with hydrogen sulphide conveniently in a solvent such as pyridine or dimethylformamide and in the presence of a base such as triethylamine and subsequently alkylating the thioamide formed with a corresponding alkyl or aralkyl halide.

c) In order to prepare a compound of general formula I wherein Ar denotes a phenyl or naphthyl group substituted by the groups R₅, R₆ and R₇, while R₆ and R₇ are as hereinbefore defined and R₅ denotes an aminomethyl, C₁₋₃-alkylaminomethyl or di-(C₁₋₃-alkyl)aminomethyl group:

Catalytic hydrogenation of a compound of general formula

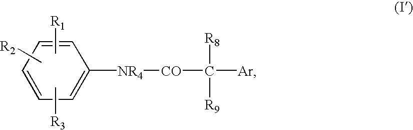

wherein

Ar denotes a phenyl or naphthyl group substituted by the groups R₅, R₆ and R₇, R₁ to R₄ and R₆ to R₉ are as hereinbefore defined and R₅ denotes a cyano group, and optionally subsequent alkylation with a compound of formula

R₁₂—Z₂ (VI),

wherein R₁₂ denotes a C₁₋₃-alkyl group and Z₂ denotes a leaving group such as a halogen atom or a sulphonyloxy group, e.g. a chlorine, bromine or iodine atom or a trifluoromethyl-sulphonyloxy group.

The catalytic hydrogenation is carried out with hydrogen in the presence of a catalyst such as palladium/charcoal, platinum in a solvent such as methanol, ethanol, ethyl acetate, dimethylformamide, dimethylformamide/acetone or glacial acetic acid optionally with the addition of an acid such as hydrochloric acid at temperatures between 0 and 50° C., but preferably at ambient temperature, and at a hydrogen pressure of 1 to 7 bar, but preferably 3 to 5 bar, or for example with Raney nickel preferably in methanolic ammonia solution. The alkylation which optionally follows is conveniently carried out in a solvent or mixture of solvents such as methylene chloride, dimethylformamide, benzene, toluene, chlorobenzene, tetrahydrofuran, benzene/tetrahydrofuran, dioxan, dimethylsulphoxide or sulpholane with an alkylating agent such as a corresponding halide or sulphonic acid ester, e.g. with methyl iodide, ethyl bromide, dimethylsulphate or benzyl chloride, optionally in the presence of a tertiary organic base or in the presence of an inorganic base conveniently at temperatures between 0 and 150° C., preferably at temperatures between 0 and 100° C.

If according to the invention a compound of general formula I is obtained which contains an amino or imino group, this may subsequently be converted with a corresponding acyl derivative into a corresponding acyl compound of general formula I and/or

if a compound of general formula I is obtained which contains an esterified carboxy group, this may be converted by hydrolysis into a corresponding carboxylic acid of general formula I and/or

if a compound of general formula I is obtained which contains a carboxy group, this may subsequently be converted by esterification into a corresponding ester.

The subsequent acylation is conveniently carried out with a corresponding halide or anhydride in a solvent such as methylene chloride, chloroform, carbon tetrachloride, ether, tetrahydrofuran, dioxan, benzene, toluene, acetonitrile or sulpholane optionally in the presence of an inorganic or organic base at temperatures between −20 and 200° C., but preferably at temperatures between −10 and 160° C. This may however also be carried out with the free acid, optionally in the presence of an acid-activating agent or a dehydrating agent, e.g. in the presence of isobutyl chloroformate, thionyl chloride, trimethylchlorosilane, hydrogen chloride, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide or 1-hydroxy-benzotriazole, N,N′-carbonyldiimidazole or N,N′-thionyldiimidazole or triphenylphosphine/carbon tetrachloride, at temperatures between −20 and 200° C., but preferably at temperatures between −10 and 160° C.

The subsequent hydrolysis is conveniently carried out either in the presence of an acid such as hydrochloric acid, sulphuric acid, phosphoric acid, acetic acid, trichloroacetic acid, trifluoroacetic acid or mixtures thereof or in the presence of a base such as lithium hydroxide, sodium hydroxide or potassium hydroxide in a suitable solvent such as water, water/methanol, water/ethanol, water/isopropanol, methanol, ethanol, water/tetrahydrofuran or water/dioxan and the subsequent decarboxylation in the presence of an acid as hereinbefore described at temperatures between −10 and 120° C., e.g. at temperatures between ambient temperature and the boiling temperature of the reaction mixture.

The subsequent esterification is carried out with a corresponding alcohol, conveniently in a solvent or mixture of solvents such as methylene chloride, benzene, toluene, chlorobenzene, tetrahydrofuran, benzene/tetrahydrofuran or dioxan, but preferably in an excess of the alcohol used, optionally in the presence of an acid such as hydrochloric acid or in the presence of a dehydrating agent, e.g. in the presence of isobutyl chloroformate, thionyl chloride, trimethylchlorosilane, hydrochloric acid, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide, N,N′-carbonyldiimidazole or N,N′-thionyldiimidazole, triphenylphosphine/carbon tetrachloride or triphenyl-phosphine/diethyl azodicarboxylate, optionally in the presence of a base such as potassium carbonate, N-ethyl-diisopropylamine or N,N-dimethylamino-pyridine, conveniently at temperatures between 0 and 150° C., preferably at temperatures between 0 and 80° C., or with a corresponding halide in a solvent such as methylene chloride, tetrahydrofuran, dioxan, dimethylsulphoxide, dimethylformamide or acetone optionally in the presence of a reaction accelerator such as sodium or potassium iodide and preferably in the presence of a base such as sodium carbonate or potassium carbonate or in the presence of a tertiary organic base such as N-ethyl-diisopropylamine or N-methyl-morpholine, which may also simultaneously serve as the solvent, or optionally in the presence of silver carbonate or silver oxide at temperatures between −30 and 100° C., but preferably at temperatures between −10 and 80° C.

In the reactions described hereinbefore, any reactive groups present such as hydroxy, carboxy, amino, alkylamino or imino groups may be protected during the reaction by conventional protecting groups which are cleaved again after the reaction.

For example, a protecting group for a hydroxy group may be a methoxy, benzyloxy, trimethylsilyl, acetyl, benzoyl, tert-butyl, trityl, benzyl or tetrahydropyranyl group,

protecting groups for a carboxy group may be a trimethylsilyl, methyl, ethyl, tert.butyl, benzyl or tetrahydropyranyl group and

protecting groups for an amino, alkylamino or imino group may be an acetyl, trifluoroacetyl, benzoyl, ethoxycarbonyl, tert-butoxycarbonyl, benzyloxycarbonyl, benzyl, methoxybenzyl or 2,4-dimethoxybenzyl group and additionally, for the amino group, a phthalyl group.

Any protecting group used is optionally subsequently cleaved for example by hydrolysis in an aqueous solvent, e.g. in water, isopropanol/water, tetrahydrofuran/water or dioxan/water, in the presence of an acid such as trifluoroacetic acid, hydrochloric acid or sulphuric acid or in the presence of an alkali metal base such as lithium hydroxide, sodium hydroxide or potassium hydroxide or by ether splitting, e.g. in the presence of iodotrimethylsilane, at temperatures between 0 and 100° C., preferably at temperatures between 10 and 50° C.

However, a benzyl, methoxybenzyl or benzyloxycarbonyl group is cleaved, for example, hydrogenolytically, e.g. with hydrogen in the presence of a catalyst such as palladium/charcoal in a solvent such as methanol, ethanol, ethyl acetate, dimethylformamide, dimethylformamide/acetone or glacial acetic acid optionally with the addition of an acid such as hydrochloric acid at temperatures between 0 and 50° C., but preferably at ambient temperature, and at a hydrogen pressure of 1 to 7 bar, but preferably 3 to 5 bar.

A methoxybenzyl group may also be cleaved in the presence of a oxidising agent such as cerium(IV)ammonium nitrate in a solvent such as methylene chloride, acetonitrile or acetonitrile/water at temperatures between 0 and 50° C., but preferably at ambient temperature.

A methoxy group is conveniently cleaved in the presence of boron tribromide in a solvent such as methylene chloride at temperatures between −35 and −25° C.

A 2,4-dimethoxybenzyl group, however, is preferably cleaved in trifluoroacetic acid in the presence of anisole.

A tert.butyl or tert.butyloxycarbonyl group is preferably cleaved by treating with an acid such as trifluoroacetic acid or hydrochloric acid, optionally using a solvent such as methylene chloride, dioxan or ether.

A phthalyl group is preferably cleaved in the presence of hydrazine or a primary amine such as methylamine, ethylamine or n-butylamine in a solvent such as methanol, ethanol, isopropanol, toluene/water or dioxan at temperatures between 20 and 50° C.

An allyloxycarbonyl group is cleaved by treating with a catalytic amount of tetrakis-(triphenylphosphine)-palladium(O), preferably in a solvent such as tetrahydrofuran and preferably in the presence of an excess of a base such as morpholine or 1,3-dimedone at temperatures between 0 and 100° C., preferably at ambient temperature and under inert gas, or by treating with a catalytic amount of tris-(triphenylphosphine)-rhodium(I)chloride in a solvent such as aqueous ethanol and optionally in the presence of a base such as 1,4-diazabicyclo[2.2.2]octane at temperatures between 20 and 70° C.

The compounds of general formulae II to VI, used as starting materials, some of which are known from the literature, are obtained by methods known from the literature and their preparation is also described in the Examples.

The chemistry of the compounds of general formula II is described, for example, by Schröter in Stickstoffverbindungen II, pages 341-730, Methoden der organischen Chemie (Houben-Weyl), 4^{th} edition, Verlag Thieme, Stuttgart 1957. The preparation of carboxylic acid derivatives of general formula III is described in Methoden der organischen Chemie (Houben-Weyl), Volume E5, Carbonsäuren und Carbonsäurederivate, 4^{th} edition, Verlag Thieme, Stuttgart 1985.

Moreover, the compounds of general formula I obtained may be resolved into their enantiomers and/or diastereomers.

Thus, for example, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical enantiomers and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be for example (+) or (−)-menthol and an optically active acyl group in amides, for example, may be a (+)- or (−)-menthyloxycarbonyl.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, if the new compounds of formula I contain a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, cyclohexylamine, ethanolamine, diethanolamine and triethanolamine.

As already mentioned, the new compounds of general formula I and the compounds 2-(5-carbamimidoyl-2-hydroxy-phenyl)-N-[3-chloro-4-(pyrrolidin-1-yl-carbonyl)-phenyl]-acetamide, 2-(5-carbamimidoyl-2-hydroxy-phenyl)-N-[3-methyl-4-(pyrrolidin-1-yl-sulphonyl)-phenyl]-acetamide, 2-(5-carbamimidoyl-2-hydroxy-phenyl)-N-[3-bromo-4-(pyrrolidin-1-yl-carbonyl)-phenyl]-acetamide and 2-(5-carbamidoyl-2-hydroxy-phenyl)-N-[3-methoxy-4-(pyrrolidin-1-yl-carbonyl)-phenyl]-acetamide and the salts thereof have valuable properties. Thus, the compounds of general formula I wherein Ar denotes a phenyl or naphthyl group substituted by the groups R₅, R₆ and R₇, and R₅ denotes a cyano group, are valuable intermediates for preparing the corresponding compounds of general formula I wherein R₅ denotes an amidino group optionally substituted by one or two C₁₋₃-alkyl groups. The compounds of general formula I with the exception of those compounds wherein Ar denotes a phenyl or naphthyl group substituted by the groups R₅, R₆ and R₇, and R₅ denotes a cyano group, as well as the tautomers, the stereoisomers and the physiologically acceptable salts thereof, have valuable pharmacological properties, particularly an antithrombotic activity which is preferably based on an effect on thrombin or factor Xa, e.g. on an inhibitory effect on thrombin or factor Xa, on a prolonging effect on aPTT time and on an inhibitory effect on related serine proteases such as e.g. trypsin, urokinase, factor VIIa, factor IX, factor XI and factor XII.

For example, the compounds

(1) 2-(3-carbamimidoyl-phenyl)-N-[3-methyl-4-(pyrrolidin-1-yl-carbonyl)-phenyl]-isobutyramide-hydrochloride,

(2) 2-(5-carbamimidoyl-2-hydroxy-phenyl)-N-[3-methyl-4-(piperidin-3-yl-carbonyl)-phenyl]-acetamide,

(3) 2-(5-carbamimidoyl-2-hydroxy-phenyl)-N-[3-chloro-4-(pyrrolidin-1-yl-carbonyl)-phenyl]-acetamide-hydrochloride and

(4) 2-(5-carbamimidoyl-2-hydroxy-phenyl)-N-[3-trifluoromethyl-4-(pyrrolidin-1-yl-carbonyl)-phenyl]-acetamide-hydrochloride

were investigated for their effect on the inhibition of factor Xa as follows:

Method: Enzyme-kinetic measurement with chromogenic substrate. The quantity of anpnitroaniline (pNA) released from the colourless chromogenic substrate by human factor Xa is determined photometrically at 405 nm. It is proportional to the activity of the enzyme used. The inhibition of the enzyme activity by the test substance (in relation to the solvent control) is determined at various concentrations of test substance and from this the IC₅₀ is calculated, as the concentration which inhibits the factor Xa used by 50%.

Material: Tris(hydroxymethyl)-aminomethane buffer (100 mmol) and sodium chloride (150 mMol), pH 8.0

Factor Xa (Roche), spec. activity: 10 U/0.5 ml, final concentration: 0.175 U/ml per reaction mixture

Substrate Chromozym X (Roche), final concentration: 200 μMol/l per reaction mixture

Test substance: final concentration 100, 30, 10, 3, 1, 0.3, 0.1, 0.03, 0.01, 0.003, 0.001 μMol/l

Procedure: 10 μl of a 23.5-times concentrated starting solution of the test substance or solvent (control), 175 μl of tris(hydroxymethyl)-aminomethane buffer and 25 μl of a 1.65 U/ml Factor Xa working solution are incubated for 10 minutes at 37° C. After the addition of 25 μl of Chromozym X working solution (1.88 μMol/l) the sample is measured in a photometer (SpectraMax 250) at 405 nm for 150 seconds at 37° C.

Evaluation

1. Determining the maximum increase (deltaOD/minutes) over 3 measuring points.

2. Determining the % inhibition based on the solvent control.

3. Plotting a dosage/activity curve (% inhibition vs substance concentration).

4. Determining the IC₅₀ by interpolating the X value (substance concentration) of the dosage/activity curve at Y=50% inhibition.

The following Table shows the results obtained:

| | Inhibition of factor Xa |
|---|---|
| Substance | (IC₅₀ in μM) |
| (1) | 0.028 |
| (2) | 0.320 |
| (3) | 0.033 |
| (4) | 0.011 |

The compounds prepared according to the invention are well tolerated, as no toxic side effects could be observed at therapeutic doses.

In view of their pharmacological properties the new compounds, with the exception of those compounds wherein Ar denotes a phenyl or naphthyl group substituted by the groups R₅, R₆ and R₇, and R₅ denotes a cyano group, and the physiologically acceptable salts thereof are suitable for the prevention and treatment of venous and arterial thrombotic diseases, such as for example the treatment of deep leg vein thrombosis, for preventing reocclusions after bypass operations or angioplasty (PT(C)A), and occlusion in peripheral arterial diseases such as pulmonary embolism, disseminated intravascular coagulation, for preventing coronary thrombosis, stroke and the occlusion of shunts. In addition, the compounds according to the invention are suitable for antithrombotic support in thrombolytic treatment, such as for example with alteplase, reteplase, tenecteplase, staphylokinase or streptokinase, for preventing long-term restenosis after PT(C)A, for the prevention and treatment of ischaemic incidents in patients with unstable angina or non-transmural cardiac infarct, for preventing metastasis and the growth of clot-dependent tumours and fibrin-dependent inflammatory processes, e.g. in the treatment of pulmonary fibrosis, for the prevention and treatment of rheumatoid arthritis, for preventing fibrin-dependent tissue adhesions and/or the formation of scar tissue and for promoting wound healing processes. The new compounds and the physiologically acceptable salts thereof may be used therapeutically in conjunction with inhibitors of platelet aggregation such as fibrinogen receptor antagonists (e.g. abciximab, eptifibatide, tirofiban), with inhibitors of ADP-induced aggregation (e.g. clopidogrel, ticlopidine), with P₂T receptor antagonists (e.g. cangrelor) or with combined thromboxane receptor antagonists/synthetase inhibitors (e.g. terbogrel).

The dosage required to achieve such an effect is appropriately 3 to 30 mg/kg, preferably 1 to 10 mg/kg by intravenous route, and 5 to 50 mg/kg, preferably 3 to 30 mg/kg by oral route, in each case administered 1 to 4 times a day. For this purpose, the compounds of formula I prepared according to the invention may be formulated, optionally together with other active substances, with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The Examples which follow are intended to illustrate the invention:

### EXAMPLE 1

### (L)-2-(5-carbamimidoyl-2-hydroxy-phenyl)-N-[3-methyl-4-(2-aminocarbonyl-pyrrolidin-1-yl-carbonyl)-phenyl]-acetamide-hydrochloride

a. 4-Allyloxy-benzonitrile

100.1 g (0.84 mol) of 4-hydroxy-benzonitrite are dissolved in 600 ml dimethylformamide and after the addition of 103.2 g (0.92 mol) of potassium tert. butoxide stirred for 30 minutes at 35° C. Then a solution of 79.6 ml (0.92 mol) of 3-bromopropene in 10 ml of dimethylformamide is added dropwise. After 1 hour at 60° C. the reaction solution is poured onto ice water, combined with 300 ml of 2 molar sodium hydroxide solution and extracted with ethyl acetate. The organic extracts are dried and evaporated down.

Yield: 130.1 g (97% of theoretical),

R_{f} value: 0.25 (silica gel; petroleum ether/ethyl acetate=8:2)

b. 3-Allyl-4-hydroxy-benzonitrile

47.8 g (0.3 mol) of 4-allyloxy-benzonitrile are heated to 210° C. under a nitrogen atmosphere for 60 minutes. After cooling the residue is chromatographed on silica gel, eluting with petroleum ether/ethyl acetate (9:1 and 1:1).

Yield: 14.7 g (31% of theoretical),

R_{f} value: 0.45 (silica gel; petroleum ether/ethyl acetate 1:1)

c. 3-Allyl-4-benzyloxy-benzonitrile

14.6 g (0.09 mol) of 3-allyl-4-hydroxy-benzonitrile and 34.6 g (0.25 mol) of potassium carbonate are stirred for 15 minutes in 200 ml dimethylformamide. After the addition of 12.1 ml (0.1 mol) of benzylbromide the reaction mixture is stirred for 2 hours at ambient temperature. The reaction solution is poured onto ice water and the crystalline product is suction filtered.

Yield: 19.9 g (87% of theoretical),

R_{f} value: 0.55 (silica gel; petroleum ether/ethyl acetate=8:2)

d. (2-benzyloxy-5-cyano)-phenylacetic acid

5.0 g (20 mmol) of 3-allyl-4-benzyloxy-benzonitrile are added in batches to a solution of 17.4 g (110 mmol) of potassium permanganate in 50 ml of water and 150 ml of glacial acetic acid, while the temperature rises to 55° C. After 1 hour at 40° C. the reaction mixture is diluted with ethyl acetate and suction filtered through Celite. The organic phase is separated off and evaporated down. The residue is chromatographed on silica gel, eluting with petroleum ether/ethyl acetate (9:1 and 1:1).

Yield: 2.2 g (40% of theoretical),

R_{f} value: 0.2 (silica gel; petroleum ether/ethyl acetate=1:1)

e. tert.butyl 4-benzylamino-2-methyl-benzoate

21.5 g (77 mmol) of tert.butyl 4-bromo-2-methyl-benzoate and 10.2 g (93.2 mmol) of benzylamine are dissolved in 250 ml of toluene and after the addition of 38 g (116.6 mmol) of caesium carbonate, 1.8 g (7.8 mmol) of palladium-II-acetate and 4.9 g (7.8 mmol) of 2,2′-bis-(diphenylphosphino)-1,1′-binaphthyl heated to 100° C. for 23 hours under a nitrogen atmosphere. After cooling the solution is filtered and evaporated down. The residue is chromatographed on silica gel, eluting with petroleum ether/ethyl acetate (95:5).

Yield: 16.7 g (72% of theoretical),

R_{f} value: 0.3 (silica gel; petroleum ether/ethyl acetate=9: 1)

f. tert.butyl 4-amino-2-methyl-benzoate

13.5 g (45 mmol) of tert.butyl 4-benzylamino-2-methyl-benzoate are dissolved in 250 ml ethanol and after the addition of 5 g of palladium on activated charcoal (10%) hydrogenated with hydrogen for 45 minutes at ambient temperature. Then the catalyst is filtered off and the filtrate is evaporated down. The residue is triturated with petroleum ether and suction filtered.

Yield: 9.2 g (97% of theoretical),

R_{f} value: 0.4 (silica gel; petroleum ether/ethyl acetate=7:3)

g. 2-(5-Cyano-2-benzyloxy-phenyl)-N-(3-methyl-4-tert.butoxycarbonyl-phenyl)-acetamide

7.5 g (28 mmol) of (2-benzyloxy-5-cyano)-phenylacetic acid are dissolved in 200 ml of tetrahydrofuran and 20 ml dimethylformamide and after the addition of 9.5 g (29 mmol) of O-(benzotriazol-1-yl)-N,N,N′,N′-tetramethyluronium tetrafluoroborate, 3.4 ml (30.6 mmol) of N-methylmorpholine and 6.1 g (29.5 mmol) of tert.butyl 4-amino-2-methyl-benzoate stirred for 7 hours at 40° C. The solvent is distilled off, the residue is dissolved in ethyl acetate and combined with petroleum ether. The product precipitated is suction filtered and dried.

Yield: 8.5 g (66% of theoretical),

R_{f} value: 0.33 (silica gel; petroleum ether/ethyl acetate=7:3)

h. 2-(5-Cyano-2-benzyloxy-phenyl)-N-(3-methyl-4-carboxy-phenyl)-acetamide

4.4 g (9.6 mmol) of 2-(5-cyano-2-benzyloxy-phenyl)-N-(3-methyl-4-tert.butoxycarbonyl-phenyl)-acetamide are dissolved in 50 ml of dichloromethane and combined with 10 ml of trifluoroacetic acid at 20° C. After 4 hours at ambient temperature the solvent is distilled off, the residue is triturated with water and suction filtered.

Yield: 3.8 g (97% of theoretical),

R_{f} value: 0.3 (silica gel; petroleum ether/ethyl acetate/glacial acetic acid=1:1:0.01)

i. (L)-2-(5-Cyano-2-benzyloxy-1phenyl)-N-[3-methyl-4-(2-aminocarbonyl-pyrrolidin-1-yl-carbonyl)-phenyl]-acetamide

1.3 g (3.2 mmol) of 2-(5-cyano-2-benzyloxy-phenyl)-N-(3-methyl-4-carboxy-phenyl)-acetamide are dissolved in 55 ml of tetrahydrofuran and after the addition of 635 mg (3.9 mmol) of N,N′-carbonyldiimidazole stirred for 1 hour at 30° C. Then 720 mg (6.4 mmol) of L-prolinamide are added at ambient temperature. After 18 hours the solvent is distilled off and the residue is chromatographed on silica gel, eluting with ethyl acetate.

Yield: 0.32 g (20% of theoretical),

R_{f} value: 0.15 (silica gel; ethyl acetate +1 drop of glacial acetic acid)

k. (L)-2-(5-carbamimidoyl-2-benzyloxy-phenyl)-N-[3-methy1-4-(2-aminocarbonyl-pyrrolidin-1-yl-carbonyl)-phenyl]-acetamide-hydrochloride

300 mg (0.6 mmol) of (L)-2-(5-cyano-2-benzyloxy-phenyl)-N-[3-methyl-4-(2-amino-carbonyl-pyrrolidin-1-yl-carbonyl)-phenyl]-acetamide are dissolved in 15 ml of saturated ethanolic hydrochloric acid and stirred for 17 hours at ambient temperature. The solvent is distilled off, the residue is dissolved in 20 ml of absolute ethanol and combined with 600 mg (6.2 mmol) of ammonium carbonate. After 22 hours the mixture is evaporated to dryness. The residue is combined with ethanol, the insoluble inorganic salts are suction filtered, the filtrate is combined with ether and the product precipitated is suction filtered.

Yield: 0.18 g (54% of theoretical),

R_{f} value: 0.5 (Reversed Phase RP 8; 5% sodium chloride solution/methanol=2:3)

### 1. (L)-2-(5-carbamimidoyl-2-hydroxy-phenyl)-N-[3-methyl-4-(2-aminocarbonyl-pyrrolidin-1-yl-carbonyl)-phenyl]-acetamide-hydrocbloride

Prepared analogously to Example 1.f. from (L)-2-(5-carbamimidoyl-2-benzyloxy-phenyl)-N-[3-methyl-4-(2-aminocarbonyl-pyrrolidin-1-yl-carbonyl)-phenyl]-acetamide-hydrochloride and palladium on activated charcoal/hydrogen in ethanol.

Yield: 74% of theoretical,

R_{f} value: 0.73 (Reversed Phase RP 8; 5% sodium chloride solution/methanol=2:3)

C₂₂H₂₅N₅O₄×HCl (423.48/459.94)

Mass spectrum: (M+H)⁺=424 (M−H)⁻=422

### EXAMPLE 2

### 2-(5-carbamimidoyl-2-hydroxy-phenyl)-N-{3-methyl-4-[2-(tert.butoxycarbonylamino-methyl)-piperidin-1-yl-carbonyl]-phenyl}-acetamide-hydrochloride

a. 2-(5-Cyano-2-benzyloxy-phenyl)-N-{3-methyl-4-[2-(tert.butyloxycarbonylamino-methyl)-piperidin-1-yl-carbonyl]-phenyl}-acetamide

Prepared analogously to Example 1.i. from 2-(5-cyano-2-benzyloxy-phenyl)-N-(3-methyl-4-carboxy-phenyl)-acetamide and 2-(tert.-butyloxycarbonylaminomethyl)-piperidine/O-(benzotriazol-1-yl)-N,N,N′,N′-tetramethyluronium tetrafluoroborate.

Yield: 79% of theoretical,

R_{f} value: 0.3 (silica gel; ethyl acetate/petroleum ether=2:3)

b. 2-(5-N-hydroxycarbamimidoyl-2-benzyloxy-phenyl)-N-{3-methyl-4-[2-(tert.butyloxy-carbonylaminomethyl)-piperidin-1-yl-carbonyl]-phenyl}-acetamide

A solution of 0.73 g (1.22 mmol) of 2-(5-cyano-2-benzyloxy-phenyl)-N-{3-methyl-4-[2-(tert.butyloxycarbonylaminomethyl)-piperidin-1-yl-carbonyl]-phenyl}-acetamide and 175 mg (2.5 mmol) of hydroxylamine hydrochloride in 50 ml of methanol is combined with a solution of 1.2 g (3.7 mmol) of caesium carbonate in 1.0 ml of water and refluxed for 18 hours. After cooling the crude product is purified on silica gel, eluting with dichloromethane/ethanol (98:2) plus 2% glacial acetic acid. The uniform fractions are combined and evaporated down.

Yield: 0.46 g (38% of theoretical),

R_{f} value: 0.46 (silica gel, dichloromethane/ethanol=1:1+1% glacial acetic acid)

c. 2-(5-carbamimidoyl-2-hydroxy-phenyl)-N-{3-methyl-4-[2-(tert.butyloxycarbonylamino-methyl)-piperidin-1-yl-carbonyl]-phenyl}-acetamide

Prepared analogously to Example 1.f. from 2-(5-N-hydroxycarbamimidoyl-2-benzyloxy-phenyl)-N-{3-methyl-4-[2-(tert.butyloxycarbonylaminomethyl)-piperidin-1-yl-carbonyl]-phenyl}-acetamide and palladium on activated charcoal (20%) in glacial acetic acid.

Yield: 25% of theoretical

R_{f} value: 0.46 (Reversed Phase RP 8; 5% sodium chloride solution/methanol=2:3)

C₂₈H₃₇N₅O₅×HCl (523.64/560.09)

Mass spectrum: (M+H)⁺=524

### EXAMPLE 3

### 2-(5-aminomethyl-2-hydroxy-phenyl)-N-[3-methyl-4-(pyrrolidin-1-yl-carbonyl)-phenyl]-acetamide

a. 2-(5-aminomethyl-2-benzyloxy-phenyl)-N-[3-methyl-4-(pyrrolidin-1-yl-carbonyl)-20 phenyl]-acetamide

300 mg (0.66 mmol) of 2-(5-cyano-2-benzyloxy-phenyl)-N-[3-methyl-4-(pyrrolidin-1-yl-carbonyl)-phenyl]-acetamide are dissolved in 30 ml of methanolic ammonia solution and after the addition of 300 mg of Raney nickel hydrogenated for 5 hours with hydrogen (5 atmospheres) at ambient temperature. The catalyst is filtered off and the filtrate is evaporated down.

Yield: 250 mg (79% of theoretical),

R_{f} value: 0.43 (silica gel; dichloromethane/ethanol=8:2+ammonia)

b. 2-(5-aminomethyl-2-hydroxy-phenyl)-N-[3-methyl-4-(pyrrolidin-1-yl-carbonyl)-30 phenyl]-acetamide

Prepared analogously to Example 1f. from 2-(5-aminomethyl-2-benzyloxy-phenyl)-N-[3-methyl-4-(pyrrolidin-1-yl-carbonyl)-phenyl]-acetamide and hydrogen/palladium on activated charcoal in methanol.

Yield: 54% of theoretical,

R_{f} value: 0.16 (silica gel; dichloromethane/ethanol 8:2)

C₂₁H₂₅N₃O₃ (367.45)

Mass spectrum: (M−H)⁻=366

### EXAMPLE 4

### 2-(3-carbamimidoyl-phenyl)-N-[3-methyl-4-(pyrrolidin-1-yl-carbonyl)-phenyl]-isobutyramide-hydrochloride

a. methyl 2-(3-cyano-phenyl)-2-methyl-propionate

A solution of 2.5 g (15.4 mmol) of 3-cyanophenylacetic acid in 5 ml dimethyl sulphoxide is added dropwise at 5° C. to a suspension of 1.9 g (48 mmol) of sodium hydride in 100 ml dimethyl sulphoxide. After 10 minutes 3 ml (47.7 mmol) of methyl iodide are added. After 1 hour at ambient temperature the reaction solution is poured onto ice water, adjusted to pH 5 with glacial acetic acid and extracted with ethyl acetate. The combined organic extracts are dried and evaporated down. The crude product is chromatographed on silica gel, eluting with dichloromethane.

Yield: 2.4 g (77% of theoretical),

R_{f} value: 0.8 (silica gel; petroleum ether/ethyl acetate=1:1)

b. 2-(3-cyano-phenyl)-2-methyl-propionic acid

2.4 g (11.5 mmol) of methyl 2-(3-cyano-phenyl)-2-methyl-propionate are dissolved in 16 ml of tetrahydrofuran, combined with a solution of 1 g (23.8 mmol) of lithium hydroxide in 20 ml of water and stirred for 20 hours at ambient temperature. The tetrahydrofuran is distilled off, the residue is adjusted to pH 4 with hydrochloric acid and extracted with ethyl acetate. The combined organic extracts are dried and evaporated down.

Yield: 1.5 g (66% of theoretical),

R_{f} value: 0.3 (silica gel; petroleum ether/ethyl acetate=1:1)

c. 2-(3-cyano-phenyl)-N-[3-methyl-4-(pyrrolidin-1-yl-carbonyl)-phenyl]-isobutyramide

Prepared analogously to Example 1.g. from 2-(3-cyano-phenyl)-2-methyl-propionic acid, O-(benzotriazol-1-yl)-N,N,N′,N′-tetramethyluronium tetrafluoroborate, N-methylmorpholine and 3-methyl-4-(pyrrolidin-1-yl-carbonyl)-aniline in dimethylformamide.

Yield: 82% of theoretical,

R_{f} value: 0.35 (silica gel; dichloromethane/ethanol=19:1)

d. 2-(3-carbamimidoyl-phenyl)-N-[3-methyl-4-(pyrrolidin-1-yl-carbonyl)-phenyl]-isobutyramide-hydrochloride

Prepared analogously to Example 1.k. from 2-(3-cyano-phenyl)-N-[3-methyl-4-(pyrrolidin-1-yl-carbonyl)-phenyl]-isobutyramide and hydrochloric acid/ammonium carbonate in ethanol.

Yield: 54% of theoretical,

R_{f} value: 0.35 (silica gel; dichloromethane/ethanol/glacial acetic acid=8:2:0.2)

C₂₃H₂₈N₄O₂×HCl (392.51/428.97)

Mass spectrum: (M+H)⁺=393

(M+Cl)⁻=427/29 (Cl)

### EXAMPLE 5

### 2-(5-carbamimidoyl-2-ethoxycarbonylmethyloxy-phenyl)-N-[3-methyl-4-(pyrrolidin-1-yl-carbonyl)-phenyl]-acetamide-hydrochloride

a. 2-(5-cyano-2-ethoxycarbonylmethyloxy-phenyl)-N-[3-methyl-4-(pyrrolidin-1-yl-carbonyl)-phenyl]-acetamide

0.3 g (0.8 mmol) of 2-(5-cyano-2-hydroxy-phenyl)-N-[3-methyl-4-(pyrrolidin-1-yl-carbonyl)-phenyl]-acetamide are dissolved in 20 ml of acetone and after the addition of 276 mg (2 mmol) of potassium carbonate stirred for 15 minutes at ambient temperature. Then 0.1 ml (0.9 mmol) of ethyl bromoacetate are added. After 1 hour at 40° C. the reaction solution is stirred in ice water and extracted with ethyl acetate. The combined organic extracts are dried, evaporated down and crystallised with ether/petroleum ether.

Yield: 0.22 g (59% of theoretical),

R_{f} value: 0.4 (silica gel; dichloromethane/ethanol=19:1)

b. 2-(5-carbamimidoyl-2-ethoxycarbonylmethyloxy-phenyl)-N-[3-methyl-4-(pyrrolidin-1-yl-carbonyl)-phenyl]-acetamide-hydrochloride

Prepared analogously to Example 1.k. from 2-(5-cyano-2-ethoxycarbonylmethyloxy-phenyl)-N-[3-methyl-4-(pyrrolidin-1-yl-carbonyl)-phenyl]-acetamide and hydrochloric acid/ammonium carbonate in ethanol.

Yield: 73% of theoretical,

R_{f} value: 0.35 (Reversed Phase RP 8; 5% sodium chloride solution/methanol 2:3)

C₂₅H₃₀N₄O₅×HCl (466.54/503.00)

Mass spectrum: (M+H)⁺=467 (M+Cl)⁻=501/03 (Cl)

### EXAMPLE 6

### 2-(5-carbamimidoyl-2-carboxymethyloxy-phenyl)-N-[3-methyl-4-(pyrrolidin-1-yl-carbonyl)-phenyl]-acetamide-hydrochloride

0.12 g (0.24 mmol) of 2-(5-carbamimidoyl-2-ethoxycarbonylmethyl-oxy-phenyl)-N-[3-methyl-4-(pyrrolidin-1-yl-carbonyl)-phenyl]-acetamide-hydrochloride are stirred in 10 ml of 6 molar hydrochloric acid for 16 hours at ambient temperature. Then the solvent is distilled off, the residue combined with acetone and ether and again evaporated to dryness.

Yield: 42 mg (37% of theoretical),

R_{f} value: 0.45 (Reversed Phase RP 8; 5% sodium chloride solution/methanol=2:3)

C₂₃H₂₆N₄O₅×HCl (438.49/474.95)

Mass spectrum: (M+H)⁺=439 (M−H)⁻=437

### EXAMPLE 7

### 2-(5-carbamimidoyl-2-hydroxy-phenyl)-N-[3-methyl-4-(piperidin-3-yl-carbonyl)-phenyl]-acetamide

a. 3-methyl-4-(pyridin-3-yl-carbonyl)-aniline

14.9 g (111.6 mmol) of aluminium chloride, 4.2 g (27.8 mmol) of 3-methylacetanilide and 9.9 g (55.8 mmol) of nicotinyl chloride-hydrochloride are stirred for 2 hours at 100 ° C. The reaction mixture is stirred into ice water while still hot and extracted with ethyl acetate. The combined organic extracts are dried and evaporated down. The residue is stirred for 1.5 hours at 100° C. with 50 ml of 6 molar hydrochloric acid. Then it is cooled, adjusted to pH 8 with ammonia and extracted with dichloromethane. The organic phase is evaporated down and purified on silica gel, eluting with dichloromethane/methanol (1-4%).

Yield: 1.4 g (24% of theoretical),

R_{f} value: 0.4 (silica gel; dichloromethane/ethanol 19:1)

b. 2-(5-cyano-2-benzyloxy-phenyl)-N-[3-methyl-4-(pyridin-3-yl-carbonyl)-phenyl]-acetamide

Prepared analogously to Example 1.g. from 3-methyl-4-(pyridin-3-yl-carbonyl)-aniline, (2-benzyloxy-5-cyanophenyl)-acetic acid, O-(benzotriazol-1-yl)-N,N,N′,N′-tetra-methyluronium tetrafluoroborate and N-methylmorpholine in dimethylformamide.

Yield: 56% of theoretical,

R_{f} value: 0.8 (silica gel; ethyl acetate/ethanol=9:1)

c. 2-(5-carbamimidoyl-2-benzyloxy-phenyl)-N-[3-methyl-4-(pyridin-3-yl-carbonyl)-phenyl]-acetamide

Prepared analogously to Example 1.k. from 2-(5-cyano-2-benzyloxy-phenyl)-N-[3-methyl-4-(pyridin-3-yl-carbonyl)-phenyl]-acetamide and hydrochloric acid/ammonium carbonate in ethanol.

Yield: 44% of theoretical,

R_{f} value: 0.2 (silica gel; dichloromethane/ethanol/glacial acetic acid=8:2:0.2)

d. 2-(5-carbamimidoyl-2-hydroxy-phenyl)-N-[3-methyl-4-(piperidin-3-yl-carbonyl)-phenyl]-acetamide

0.32 g (0.6 mmol) of 2-(5-carbamimidoyl-2-benzyloxy-phenyl)-N-[3-methyl-4-(pyridin-3-yl-carbonyl)-phenyl]-acetamide are dissolved in 75 ml methanol, adjusted to pH 8 with 0.5 ml conc. ammonia and after the addition of 0.15 g palladium on activated charcoal (10%) hydrogenated for 70 minutes at ambient temperature with hydrogen. Then the catalyst is filtered off and the filtrate is evaporated down. The residue is stirred with ethyl acetate/ether/petroleum ether (1:1:1) and the crystalline product is suction filtered.

Yield: 0.13 g (55% of theoretical),

R_{f} value: 0.7 (Reversed Phase RP 8; 5% sodium chloride solution/methanol=1:1)

C₂₂H₂₆N₄O₃ (394.47)

Mass spectrum: (M+H)⁺=395 (M−H)⁻=393

The following compounds are prepared analogously to Example 7:

### (1) 2-(5-carbamimidoyl-2-hydroxy-phenyl)-N-(3-methyl-4-benzoyl-phenyl)-acetamide-hydrochloride

Yield: 12% of theoretical,

R_{f} value: 0.4 (silica gel; dichloromethane/ethanol=7:3+1% glacial acetic acid)

C₂₃H₂₁N₃O₃×HCl (387.44/423.90)

Mass spectrum: (M+H)⁺=388

(M−H)⁻=386

### (2) 2-(5-carbamimidoyl-2-hydroxy-phenyl)-N-[3-methyl-4-(1-hydroxy-1-phenyl-methyl)-phenyl]-acetamide-hydrochloride

Yield: 23% of theoretical,

R_{f} value: 0.35 (silica gel; dichloromethane/ethanol=7:3+1% glacial acetic acid)

C₂₃H₂₃N₃O₃×HCl (389.46/425.92)

Mass spectrum: (M+H)⁺=390 (M−H)⁻=388

### (3) 2-(5-carbamimidoyl-2-hydroxy-phenyl)-N-{4-[1-(3-carbamimidoyl-phenyl)-1-hydroxy-methyl]-3-methyl-phenyl}-acetamide-dihydrochloride

Yield: 37% of theoretical,

R_{f} value: 0.25 (Reversed Phase RP 8; 5% sodium chloride solution/methanol=2:3)

C₂₄H₂₅N₅O₃×2 HCl (431.50/504.42)

Mass spectrum: (M+H)⁺=432

### (4) 2-(3-carbamidoyl-phenyl)-N-[3-methyl-4-(pyridin-3-yl-carbonyl)-phenyl]-isobutyramide-hydrochloride

Yield: 48% of theoretical,

R_{f} value: 0.20 (silica gel; dichloromethane/ethanol=8:2+1% glacial acetic acid)

C₂₄H₂₄N₄O₂×HCl (400.48/436.94)

Mass spectrum: (M+H)⁺=401 (M+Cl)⁻=435/37 (Cl)

### EXAMPLE 8

### 2-(5-carbamimidoyl-2-hydroxy-phenyl)-N-[3-methyl-4-(pyrrolidin-1-yl-carbonyl)-phenyl]-isobutyramide-hydrochloride

a. 3-(1,1-Dimethyl-allyl)-4-hydroxy-benzonitrile

25 g (0.13 mol) of 4-(3-methyl-but-2-enyloxy)-benzonitrile are refluxed in 250 ml dimethylformamide for 90 hours. The solvent is distilled off and the residue is taken up in ethyl acetate/water. The organic phase is dried and evaporated down. The residue is dissolved in toluene and extracted with 2 molar sodium hydroxide solution. The aqueous phase is filtered through activated charcoal and acidified with glacial acetic acid. The precipitate formed is suction filtered and dried.

Yield: 1.8 g (7% of theoretical),

R_{f} value: 0.53 (silica gel; petroleum ether/ethyl acetate=7:3)

b. 4-benzyloxy-3-(1,1-dimethyl-allyl)-benzonitrile

Prepared analogously to Example 1.c. from 3-(1,1-dimethyl-allyl)-4-hydroxy-benzonitrile, benzyl bromide and potassium carbonate in dimethylformamide.

Yield: 93% of theoretical,

R_{f} value: 0.70 (silica gel; petroleum ether/ethyl acetate =7:3)

c. 2-(2-benzyloxy-5-cyano-phenyl)-2-methyl-propionic acid

2.2 g (7.9 mmol) of 4-benzyloxy-3-(l,1-dimethyl-allyl)-benzonitrile are dissolved in 60 ml acetonitrile and after the addition of a solution of 11.9 g (55.6 mmol) of sodium metaperiodate and 50 mg (0.24 mmol) of ruthenium-(III)-chloride in 80 ml of water stirred for 4.5 hours at 45° C. After dilution with ethyl acetate, the organic phase is separated off and evaporated down. The residue is dissolved in dichloromethane and extracted with 1 molar sodium hydroxide solution. The aqueous phase is filtered through activated charcoal, the filtrate is poured onto 400 ml of 6 molar hydrochloric acid and 200 g of ice. The precipitate is suction filtered and dried.

Yield: 0.95 g (41% of theoretical),

R_{f} value: 0.22 (silica gel; petroleum ether/ethyl acetate/glacial acetic acid=7:3:0.1)

d. 2-(2-benzyloxy-5-cyano-phenyl)-2-methyl-propionic acid chloride

510 mg (1.73 mmol) of 2-(2-benzyloxy-5-cyano-phenyl)-2-methyl-propionic acid are dissolved in 10 ml dichloromethane and after the addition of 0.38 ml (5.21 mmol) of thionyl chloride and 0.1 ml of dimethylformamide heated to 50° C. for 2.5 hours. Then the reaction mixture is evaporated down and further reacted without purification.

Yield: 530 mg (98% of theoretical).

e. 2-(2-benzyloxy-5-cyano-phenyl)-N-[3-methyl-4-(pyrrolidin-1-yl-carbonyl)-phenyl]-isobutyramide

A solution of 390 mg (1.9 mmol) of 3-methyl-4-(pyrrolidin-1-yl-carbonyl)-aniline in 50 ml of tetrahydrofuran is combined with 0.7 ml (5.18 mmol) of triethylamine. Then a solution of 530 mg (1.69 mmol) of 2-(2-benzyloxy-5-cyano-phenyl)-2-methyl-propionic acid chloride in 20 ml of tetrahydrofuran is added dropwise. After 14 hours at ambient temperature the solvent is evaporated off and the residue is purified on silica gel, eluting with ethyl acetate/petroleum ether (4:1).

Yield: 410 mg (50% of theoretical),

R_{f} value: 0.38 (silica gel; ethyl acetate)

f. 2-(5-carbamimidoyl-2-hydroxy-phenyl)-N-[3-methyl-4-(pyrrolidin-1-yl-carbonyl)-phenyl]-isobutyramide-hydrochloride

Prepared analogously to Example 1.k. from 2-(2-benzyloxy-5-cyano-phenyl)-N-[3-methyl-4-(pyrrolidin-1-yl-carbonyl)-phenyl]-isobutyramide and hydrochloric acid/ammonium carbonate in ethanol followed by reaction analogously to Example 1.1. with hydrogen/palladium on activated charcoal in methanol.

Yield: 77% of theoretical,

R_{f} value: 0.5 (Reversed Phase RP 8; 5% sodium chloride solution/methanol 2:3)

C₂₃H2₈N₄O₃×HCl (408.50/444.96)

Mass spectrum: (M+H)⁺=409 (M−H)⁻=407

### EXAMPLE 9

### 2-(5-carbamimidoyl-2-hydroxy-phenyl)-N-[3-chloro-4-(pyrrolidin-1-yl-carbonyl)-phenyl]-acetamide-hydrochloride

a. 3-chloro-4-(pyrrolidin-1-yl-carbonyl)-nitrobenzene

Prepared analogously to Example 1.g. from 2-chloro-4-nitrobenzoic acid, O-(benzotriazol-1-yl)-N,N,N′-N′-tetramethyluronium fluoroborate, N-ethyl-diisopropylamine and pyrrolidine in tetrahydrofuran/water 9:1.

Yield: 72% of theoretical,

R_{f} value: 0.65 (silica gel; dichloromethane/ethanol=95:5)

b. 3-chloro-4-(pyrrolidin-1-yl-carbonyl)-aniline

Prepared analogously to Example 1.f. from 3-chloro-4-(pyrrolidin-1-yl-carbonyl)-nitrophenol and hydrogen/palladium on activated charcoal in methanol/dichloromethane (1:1).

Yield: 100% of theoretical,

R_{f} value: 0.42 (silica gel; dichloromethane/ethanol=95:5)

c. 2-(2-benzyloxy-5-cyano-phenyl)-N-[3-chloro-4-(pyrrolidin-1-yl-carbonyl)-phenyl]-acetamide

Prepared analogously to Example 8.e. from 3-chloro-4-(pyrrolidin-1-yl-carbonyl)-aniline, (2-benzyloxy-5-cyanophenyl)-acetic acid chloride and triethylamine in tetrahydrofuran.

Yield: 64% of theoretical,

R_{f} value: 0.75 (silica gel; dichloromethane/ethanol=95:5)

d. 2-(5-carbamimidoyl-2-hydroxy-phenyl)-N-[3-chloro-4-(pyrrolidin-1-yl-carbonyl)-phenyl]-acetamide-hydrochloride

Prepared analogously to Example 1.k. from 2-(2-benzyloxy-5-cyano-phenyl)-N-[3-chloro-4-(pyrrolidin-1-yl-carbonyl)-phenyl]-acetamide and hydrochloric acid/ammonium carbonate in ethanol followed by reaction analogously to Example 1.1. with hydrogen/palladium on activated charcoal in methanol.

Yield: 50% of theoretical,

R_{f} value: 0.5 (Reversed Phase RP 8; 5% sodium chloride solution/methanol=2:3)

C₂₀H₂₁CIN₄O₃×HCl (400.87/437.33)

Mass spectrum: (M+H)⁺=401 (M−H)⁻=399 (M+Cl)⁻=435/37 (Cl)

### EXAMPLE 10

### 2-(3-carbamimidoyl-phenyl)-N-[3-methyl-4-(pyrrolidin-1-yl-carbonyl)-phenyl]-2-amino-acetamide-dihydrochloride

a. 5-(3-bromophenyl)-imidazolidin-2,4-dione

A mixture of 68.0 g (0.58 mol) of 3-bromobenzaldehyde, 43.0 g (0.66 mol) of potassium cyanide and 259 g (2.7 mol) of ammonium carbonate are suspended in 1 l of water and 1 l of ethanol and stirred for 1 h at 60° C. Then the suspension is cooled and carefully adjusted to pH 4 with 665 ml conc. hydrochloric acid. The suspension formed is cooled to 5° C., suction filtered, washed with water and dried.

Yield: 100.6 g (68% of theoretical),

R_{f} value: 0.5 (silica gel; dichloromethane/ethanol=9.5:0.5)

b. amino-(3-bromophenyl)-acetic acid

100 g (0.39 mol) of 5-(3-bromophenyl)-imidazolidin-2,4-dione are suspended in 170 ml of water and 295 ml (5.4 mol) of conc. sulphuric acid are added dropwise within 25 minutes, during which time the temperature rises to 85° C. Then the reaction mixture is heated to 150° C. for 6.5 h. After cooling, 10 g of activated charcoal are added and the mixture is suction filtered. The solution is poured onto ice water, adjusted to pH 6.5 with about 650 ml of conc. ammonia and the precipitate formed is suction filtered.

Yield: 83.6 g (93% of theoretical),

R_{f} value: 0.6 (Reversed Phase RP 8; 5% sodium chloride solution/methanol=2:3)

c. ethyl amino-(3-bromophenyl)-acetate-hydrochloride

Prepared analogously to Example 6 from amino-(3-bromophenyl)-acetic acid and 6 molar hydrochloric acid in ethanol.

Yield: 95% of theoretical,

R_{f} value: 0.45 (Reversed Phase RP 8; 5% sodium chloride solution/methanol=2:3)

d. (3-bromophenyl)-tert.butoxycarbonylamino-acetic acid

12.1 g (41 mmol) of ethyl amino-(3-bromophenyl)-acetate-hydrochloride are dissolved in 90.4 ml (90.4 mmol) of 1 molar sodium hydroxide solution. After the addition of a solution of 9.0 g (41 mmol) of di-tert.butyldicarbonate in 100 ml of tetrahydrofuran the reaction mixture is stirred for 18 hours at ambient temperature. Then it is extracted with ether. The aqueous phase is adjusted to pH 1 with 1 molar hydrochloric acid and extracted with ether. The combined organic extracts are dried and evaporated down.

Yield: 12.0 g (89% of theoretical),

R_{f} value: 0.15 (silica gel; dichloromethane/ethanol=95:5)

e. benzyl (3-bromophenyl)-tert.butoxycarbonylamino-acetate

Prepared analogously to Example 1.c. from (3-bromophenyl)-tert.butoxycarbonylamino-acetic acid, benzyl bromide and potassium carbonate in dimethylformamide.

Yield: 85% of theoretical,

R_{f} value: 0.24 (silica gel; petroleum ether/ethyl acetate=9:1)

f. benzyl tert.butoxycarbonylamino-(3-cyanophenyl)-acetate

11.4 g (27 mmol) of benzyl (3-bromophenyl)-tert.butoxycarbonylamino-acetate are dissolved in 100 ml dimethylformamide and after the addition of 4.8 g (53.6 mmol) of copper-(I)-cyanide and 0.7 g (0.6 mmol) of tetrakis-triphenylphosphine-palladium-(O) stirred for 8 hours at 145° C. The warm suspension is suction filtered and the mother liquor is distributed in sodium chloride solution/ethyl acetate. The aqueous phase is extracted three times with ethyl acetate, the combined organic extracts are washed with sodium hydroxide solution, dried, evaporated down and chromatographed on silica gel, eluting with petroleum ether/ethyl acetate (15-25%).

Yield: 3.7 g (37% of theoretical),

R_{f} value: 0.25 (silica gel; dichloromethane/ethanol=4:1)

g. tert.-Butoxycarbonylamino-(3-cyanophenyl)-acetic acid

Prepared analogously to Example 1.f. from benzyl tert.-butoxycarbonylamino-(3-cyanophenyl)-acetate and palladium on activated charcoal/hydrogen in methanol.

Yield: 100% of theoretical,

R_{f} value: 0.2 (silica gel; dichloromethane/ethanol=95:5)

h. 2-(3-cyano-phenyl)-N-[3-methyl-4-(pyrrolidin-1-yl-carbonyl)-phenyl]-2-(tert.butoxy-carbonylamino)-acetamide

0.7 g (2.5 mmol) of tert.-butoxycarbonylamino-(3-cyanophenyl)-acetic acid, 0.5 g (2.5 mmol) of 3-methyl-4-(pyrrolidin-1-yl-carbonyl)-aniline and 1.7 ml (15.2 mmol) of N-methylmorpholine are dissolved in 30 ml dichloromethane and at −10° C. combined with 3.0 ml (5 mmol) of propanephosphonic acid-cycloanhydride. The reaction mixture is stirred for 1 h at −10° C. and for 48 hours at ambient temperature. The solvent is distilled off, the residue is taken up in ethyl acetate, then washed with sodium hydroxide solution and water. The organic phases are dried and evaporated down.

Yield: 0.8 g (64% of theoretical),

R_{f} value: 0.3 (silica gel; dichloromethane/ethanol=95:5)

C₂₆H₃₀N₄O₄ (462.55)

Mass spectrum: (M+H)⁺=463 (M−H)⁻=461

i. 2-(3-carbamimidoyl-phenyl)-N-[3-methyl-4-(pyrrolidin-1-yl-carbonyl)-phenyl]-2-amino-acetamide-dihydrochloride

Prepared analogously to Example 1.k. from 2-(3-cyano-phenyl)-N-[3-methyl-4-(pyrrolidin-1-yl-carbonyl)-phenyl]-2-(tert.butoxycarbonylamino)-acetamide and hydrochloric acid/ammonium carbonate in ethanol.

Yield: 100% of theoretical,

R_{f} value: 0.2 (silica gel; dichloromethane/ethanol=4:1+5% ammonia)

C₂₁H₂₅N₅O₂×2 HCl (379.46/452.39)

Mass spectrum: (M+H)⁺=380

The following compound is prepared analogously to Example 10:

### (1) 2-(3-carbamimidoyl-phenyl)-N-[3-methyl-4-(pyrrolidin-1-yl-carbonyl)-phenyl]-2-(acetylamino)-acetamide-hydrochloride

Yield: 96% of theoretical,

R_{f} value: 0.59 (Reversed Phase RP 8; 5% sodium chloride solution/methanol=2:3)

C₂₃H₂₇N₅O₃×HCl (421.5/457.95)

Mass spectrum: (M+H)⁺=422 (M−H)⁻=420

### EXAMPLE 11

### 2-(5-carbamimidoyl-2-hydroxy-phenyl)-N-[3-methyl-4-(pyrrolidin-1-yl-sulphonyl)-phenyl]-acetamide-hydrochloride

a. 4-Acetylamino-2-methyl-benzenesulphonic acid chloride

5.0 g (33.5 mmol) of 3-methyl-phenylacetamide are added batchwise at ambient temperature to 11.1 ml (167 mmol) of chlorosulphonic acid. The reaction mixture is heated to 60° C. for 2 hours, cooled, poured onto ice water and extracted with ethyl acetate. The combined organic extracts are dried and evaporated down.

Yield: 7.0 g (85% of theoretical),

R_{f} value: 0.2 (silica gel; dichloromethane/ethanol 9:1)

b. N-[3-methyl-4-(pyrrolidin-1-yl-sulphonyl)-phenyl]-acetamide

7.0 g (28.4 mmol) of 4-acetylamino-2-methyl-benzenesulphonyl chloride are suspended in 70 ml of water and 60 ml of 0.5 molar sodium hydroxide solution and at 0° C. combined with a solution of 2.5 ml (29.8 mmol) of pyrrolidine in 60 ml acetone. After 12 hours at ambient temperature the solution formed is acidified with 2 molar hydrochloric acid and extracted with ethyl acetate. The combined organic extracts are dried and evaporated down.

Yield: 7.2 g (89% of theoretical),

R_{f} value: 0.55 (silica gel; dichloromethane/ethanol=9:1)

c. 3-methyl-4-(pyrrolidin-1-yl-sulphonyl)-aniline

7.2 g (25.3 mmol) of N-[3-methyl-4-(pyrrolidin-1-yl-sulphonyl)-phenyl]-acetamide are dissolved in 10 ml ethanol and after the addition of 70 ml of 6 molar hydrochloric acid stirred for 11 hours at 40° C. Then the mixture is extracted several times with dichloromethane, the combined organic extracts are washed with sodium hydrogen carbonate solution, dried and evaporated down.

Yield: 3.8 g (63% of theoretical),

R_{f} value: 0.80 (silica gel; dichloromethane/ethanol=9:1)

d. 2-(2-benzyloxy-5-cyano-phenyl)-N-[3-methyl-4-(pyrrolidin-1-yl-sulphonyl)-phenyl)-acetamide

Prepared analogously to Example 8.e. from 3-methyl-4-(pyrrolidin-1-yl-sulphonyl)-aniline, 2-benzyloxy-5-cyano-phenylacetic acid chloride and triethylamine in tetrahydrofuran.

Yield: 81% of theoretical,

R_{f} value: 0.79 (silica gel; dichloromethane/ethanol=9:1)

e. 2-(5-carbamimidoyl-2-hydroxy-phenyl)-N-[3-methyl-4-(pyrrolidin-1-yl-sulphonyl)-phenyl]-acetamide-hydrochloride

Prepared analogously to Example 1.k. from 2-(2-benzyloxy-5-cyano-phenyl)-N-[3-methyl-4-(pyrrolidin-1-yl-sulphonyl)-phenyl)-acetamide and hydrochloric acid/ammonium carbonate in ethanol followed by reaction with hydrogen/palladium on activated charcoal in methanol analogously to Example 1.1.

Yield: 56% of theoretical,

R_{f} value: 0.57 (Reversed Phase RP 8; 5% sodium chloride solution/methanol=2:3)

C₂₀H₂₄N₄O₄×HCl (416.50/452.97)

Mass spectrum: (M+H)⁺=417 (M)⁻=415 (M+Cl)⁻=451/53 (Cl)

### EXAMPLE 12

### 2-(5-carbamimidoyl-2-hydroxy-phenyl)-N-[3-methyl-4-(pyrrolidin-1-yl-carbonyl)-phenyl]-propionamide-hydrochloride

a. methyl(2-benzyloxy-5-cyano-phenyl)-acetate

Prepared analogously to Example 8.d. from 2-benzyloxy-5-cyano-phenylacetic acid and thionyl chloride in dichloromethane followed by reaction with methanol.

Yield: 85% of theoretical,

R_{f} value: 0.7 (silica gel; ethyl acetate/petroleum ether=1:1)

b. methyl 2-(2-benzyloxy-5-cyano-phenyl)-propionate

Prepared analogously to Example 4.a. from methyl 2-benzyloxy-5-cyano-phenyl-acetate, sodium hydride and methyl iodide in tetrahydrofuran.

Yield: 78% of theoretical,

R_{f} value: 0.53 (silica gel; dichloromethane/ethyl acetate=7:3)

c. 2-(2-benzyloxy-5-cyano-phenyl)-propionic acid

Prepared analogously to Example 4.b. from methyl 2-(2-benzyloxy-5-cyano-phenyl)-propionate and sodium hydroxide solution in methanol.

Yield: 93% of theoretical,

R_{f} value: 0.22 (silica gel; petroleum ether/ethyl acetate/glacial acetic acid=7:3:0.1)

d. 2-(2-benzyloxy-5-cyano-phenyl)-propionic acid chloride

Prepared analogously to Example 8.d. from 2-(2-benzyloxy-5-cyano-phenyl)-propionic acid and thionyl chloride in dichloromethane.

Yield: 96% of theoretical

e. 2-(2-benzyloxy-5-cyano-phenyl)-N-[3-methyl-4-(pyrrolidin-1-yl-carbonyl)-phenyl]-propionamide

Prepared analogously to Example 8.e. from 3-methyl-4-(pyrrolidin-1-yl-carbonyl)-aniline, 2-(2-benzyloxy-5-cyano-phenyl)-propionic acid chloride and triethylamine in tetrahydrofuran.

Yield: 97% of theoretical,

R_{f} value: 0.4 (silica gel; ethyl acetate)

f. 2-(5-carbamimidoyl-2-hydroxy-phenyl)-N-[3-methyl-4-(pyrrolidin-1-yl-carbonyl)-phenyl]-propionamide-hydrochloride

Prepared analogously to Example 1.k. from 2-(2-benzyloxy-5-cyano-phenyl)-N-[3-methyl-4-(pyrrolidin-1-yl-carbonyl)-phenyl]-propionamide and hydrochloric acid/ammonium carbonate in ethanol followed by reaction with hydrogen/palladium on activated charcoal in methanol analogously to Example 1.1.

Yield: 62% of theoretical,

R_{f} value: 0.45 (Reversed Phase RP 8; 5% sodium chloride solution/methanol 2:3)

C₂₂H₂₆N₄O₃×HCl (394.48/430.95)

Mass spectrum: (M+H)⁺=395 (M−H)⁻=393

### EXAMPLE 13

### 2-(5-carbamimidoyl-2-hydroxy-phenyl)-N-[3-trifluoromethyl-4-(pyrrolidin-1-yl-carbonyl)-phenyl]-acetamide-hydrochloride

a. 3-trifluoromethyl-4-(pyrrolidin-1-yl-carbonyl)nitrobenzene

Prepared analogously to Example 1.g. from 4-nitro-2-trifluoromethyl-benzoic acid, pyrrolidine, O-(benzotriazol-1-yl)-N,N,N′,N′-tetramethyluronium tetrafluoroborate and N-methylmorpholine in dimethylformamide.

Yield: 69% of theoretical,

R_{f} value: 0.6 (silica gel; dichloromethane/ethanol=9:1)

b. 3-trifluoromethyl-4-(pyrrolidin-1-yl-carbonyl)-aniline 1.4 g (4 mmol) of 3-trifluoromethyl-4-(pyrrolidin-1-yl-carbonyl)-nitrobenzene are dissolved in 25 ml of ethyl acetate and 25 ml of methanol and after the addition of 0.5 g of palladium on activated charcoal (10%) hydrogenated for 45 minutes at ambient temperature with hydrogen. The catalyst is filtered off and the solution is evaporated down. The crude product is recrystallised from ethyl acetate.

Yield: 0.6 g (60% of theoretical),

R_{f} value: 0.45 (silica gel; dichloromethane/ethanol=9:1)

c. 2-(2-benzyloxy-5-cyano-phenyl)-N-[3-trifluoromethyl-4-(pyrrolidin-1-1-carbonyl)-phenyl]-acetamide

Prepared analogously to Example 1.g. from 3-trifluoromethyl-4-(pyrrolidin-1-yl-carbonyl)-aniline, 2-benzyloxy-5-cyano-phenylacetic acid, O-(benzotriazol-1-yl)-N,N,N′,N′-tetramethyluronium tetrafluoroborate and N-methylmorpholine in dimethylformamide.

Yield: 32% of theoretical,

R_{f} value: 0.5 (silica gel; dichloromethane/ethanol=9:1)

d. 2-(5-carbamimidoyl-2-hydroxy-phenyl)-N-[3-trifluoromethyl-4-(pyrrolidin-1-yl-carbonyl)-phenyl]-acetamide-hydrochloride

Prepared analogously to Example 1.k. from 2-(2-benzyloxy-5-cyano-phenyl)-N-[3-trifluoromethyl-4-(pyrrolidin-1-yl-carbonyl)-phenyl]-acetamide and hydrochloric acid/ammonium carbonate in ethanol followed by reaction with hydrogen/palladium on activated charcoal in methanol analogously to Example 1.1.

Yield: 35% of theoretical,

R_{f} value: 0.25 (Reversed Phase RP 8; 5% sodium chloride solution/methanol=2:3)

C₂₁H₂₁F₃N₄O₃×HCl (434.42/470.89)

Mass spectrum: (M+H)⁺=435 (M−H)⁻=433

The following compound is prepared analogously to Example 13:

### (1) 2-(5-carbamimidoyl-2-hydroxy-phenyl)-N-[4-(pyrrolidin-1-yl-carbonyl)-3-trifluoromethyl-phenyl]-propionamide-hydrochloride

Yield: 50% of theoretical,

R_{f} value: 0.24 (Reversed Phase RP 8; 5% sodium chloride solution/methanol=2:3)

C₂₂H₂₃F₃N₄O₃×HCl (448.45/484.92)

Mass spectrum: (M+H)⁺=449 (M+H)⁻=447 (M+Cl)⁻=483/85 (Cl)

### EXAMPLE 14

### 2-(5-carbamimidoyl-2-hydroxy-phenyl)-N-[3-bromo-4-(pyrrolidin-1-yl-carbonyl)-phenyl]-acetamide-hydrochloride

a. 3-bromo-4-(pyrrolidin-1-yl-carbonyl)-nitrobenzene

Prepared analogously to Example 1.g. from 2-bromo-4-nitro-benzoic acid, pyrrolidine, O-(benzotriazol-1-yl)-N,N,N′,N′-tetramethyluronium tetrafluoroborate and N-methylmorpholine in dimethylformamide.

Yield: 77% of theoretical,

R_{f} value: 0.25 (silica gel; petroleum ether/ethyl acetate=1:1)

b. 3-bromo-4-(pyrrolidine-1-yl-carbonyl)-aniline

Prepared analogously to Example 13.b. from 3-bromo-4-(pyrrolidin-1-yl-carbonyl)-nitrobenzene and hydrogen/palladium on activated charcoal in ethyl acetate.

Yield: 28% of theoretical,

R_{f} value: 0.33 (silica gel; ethyl acetate/petroleum ether=9:1+1 drop of ammonia)

c. 2-(2-benzyloxy-5-cyano-phenyl)-N-[3-bromo-4-(pyrrolidin-1-yl-carbonyl)-phenyl]-acetamide

Prepared analogously to Example 1.g. from 3-bromo-4-(pyrrolidin-1-yl-carbonyl)-aniline, 2-benzyloxy-5-cyano-phenylacetic acid, O-(benzotriazol-1-yl)-N,N,N′,N′-tetramethyl-uronium tetrafluoroborate and N-methylmorpholine in dimethylformamide.

Yield: 53% of theoretical,

R_{f} value: 0.4 (silica gel; ethyl acetate)

d. 2-(5-carbamimidoyl-2-hydroxy-phenyl)-N-[3-bromo-4-(pyrrolidin-1-yl-carbonyl)-phenyl]-acetamide-hydrochloride

Prepared analogously to Example 1.k. from 2-(2-benzyloxy-5-cyano-phenyl)-N-[3-bromo-4-(pyrrolidin-1-yl-carbonyl)-phenyl]-acetamide and hydrochloric acid/ammonium carbonate in ethanol followed by reaction with hydrogen/palladium on activated charcoal in methanol analogously to Example 1.1.

Yield: 46% of theoretical,

R_{f} value: 0.5 (Reversed Phase RP 8; 5% sodium chloride solution/methanol 2:3)

C₂₀H₂₁BrN₄O₃×HCl (445.32/481.79)

Mass spectrum: (M+H)⁺=445/47 (Br)

The following compound is prepared analogously to Example 14:

### (1) 2-(5-carbamimidoyl-2-hydroxy-phenyl)-N-[3-bromo-4-(pyrrolidin-1-yl-carbonyl)-phenyl]-propionamide-hydrochloride

Yield: 20% of theoretical,

R_{f} value: 0.4 (Reversed Phase RP 8; 5% sodium chloride solution/methanol=2:3)

C₂₁H₂₃BrN₄O₃×HCl (459.35/495.81)

Mass spectrum: (M+H)⁺=459/61 (Br)

### EXAMPLE 15

### 2-(5-carbamidoyl-2-hydroxy-phenyl)-N-[3-methoxy-4-(pyrrolidin-1-yl-carbonyl)-phenyl]-acetamide-hydrochloride

a. 3-methoxy-4-(pyrrolidin-1-yl-carbonyl)-nitrobenzene

Prepared analogously to Example 1.g. from 2-methoxy-4-nitro-benzoic acid, pyrrolidine, O-(benzotriazol-1-yl)-N,N,N′,N′-tetramethyluronium tetrafluoroborate and N-methylmorpholine in dimethylformamide.

Yield: 70% of theoretical,

R_{f} value: 0.45 (silica gel; dichloromethane/ethanol=19:1)

b. 3-methoxy-4-(pyrrolidin-1-yl-carbonyl)-aniline

Prepared analogously to Example 13.b. from 3-methoxy-4-(pyrrolidin-1-yl-carbonyl)-nitrobenzene and hydrogen/palladium on activated charcoal in methanol.

Yield: 75% of theoretical,

R_{f} value: 0.25 (silica gel; dichloromethane/ethanol=19:1)

c. 2-(2-benzyloxy-5-cyano-phenyl)-N-[3-methoxy-4-(pyrrolidin-1-yl-carbonyl)-phenyl]-acetamide

Prepared analogously to Example 1.g. from 3-methoxy-4-(pyrrolidin-1-yl-carbonyl)-aniline, 2-benzyloxy-5-cyano-phenylacetic acid, O-(benzotriazol-1-yl)-N,N,N′,N′-tetramethyluronium tetrafluoroborate and N-methylmorpholine in dimethylformamide.

Yield: 22% of theoretical,

R_{f} value: 0.45 (silica gel; dichloromethane/ethanol 19:1)

d. 2-(5-carbamidoyl-2-hydroxy-phenyl)-N-[3-methoxy-4-(pyrrolidin-1-yl-carbonyl)-phenyl]-acetamide-hydrochloride

Prepared analogously to Example 1.k. from 2-(2-benzyloxy-5-cyano-phenyl)-N-[3-methoxy-4-(pyrrolidin-1-yl-carbonyl)-phenyl]-acetamide and hydrochloric acid/ammonium carbonate in ethanol followed by reaction with hydrogen/palladium on activated charcoal in methanol analogously to Example 1.1.

Yield: 22% of theoretical,

R_{f} value: 0.25 (Reversed Phase RP 8; 5% sodium chloride solution/methanol=2:3)

C₂₁H₂₄N₄O₄×HCl (396.45/432.92)

Mass spectrum: (M+H)⁺=397 (M−H)⁻=395 (M+Cl)⁻=431/33 (Cl)

### EXAMPLE 16

### 2-(3-carbamimidoyl-phenyl)-N-[3-bromo-4-(pyrrolidin-1-yl-carbonyl)-phenyl]-3-phenyl-propionamide-hydrochloride

a. 2-(3-cyano-phenyl)-N-[3-bromo-4-(pyrrolidin-1-yl-carbonyl)-phenyl]-3-phenyl-propionamide

0.2 g (2.0 mmol) of potassium tert. butoxide is added batchwise to a solution of 0.4 g (1.0 mmol) of 2-(3-cyano-phenyl)-N-[3-bromo-4-(pyrrolidin-1-yl-carbonyl)-phenyl]-acetamide in 4 ml of dimethylsulphoxide at ambient temperature and the mixture is stirred for 15 minutes. After the addition of 0.1 ml (1.0 mmol) of benzyl bromide the reaction mixture is stirred for a further 2 hours at ambient temperature and combined with ice water. The precipitate formed is suction filtered, dried and purified on silica gel, eluting with petroleum ether/ethyl acetate (1/0 to 0/1). The uniform fractions are combined and evaporated down.

Yield: 0.1 g (22% of theoretical)

R_{f} value: 0.46 (silica gel; ethyl acetate+1% ammonia)

b. 2-(3-carbamimidoyl-phenyl)-N-[3-bromo-4-(pyrrolidin-1-yl-carbonyl)-phenyl]-3-phenyl-propionamide-hydrochloride

Prepared analogously to Example 1.k. from 2-(3-cyano-phenyl)-N-[3-bromo-4-(pyrrolidin-1-yl-carbonyl)-phenyl-3-phenyl-propionamide and hydrochloric acid/ammonium carbonate in ethanol.

Yield: 81% of theoretical,

R_{f} value: 0.36 (Reversed Phase RP 8; 5% sodium chloride solution/methanol=1:2)

C₂₇H₂₇BrN₄O₂×HCl (519.36/555.91) Mass spectrum: (M+H)⁺=519/21 (Br) (M−H)⁻=517/19 (Br)

The following are prepared analogously to Example 16:

### (1) 2-(3-carbamimidoyl-phenyl)-N-[3-bromo-4-(pyrrolidin-1-yl-carbonyl)-phenyl]-3-(pyridin-4-yl)-propionamide-hydrochloride

Yield: 95% of theoretical,

R_{f} value: 0.48 (Reversed Phase RP 8; 5% sodium chloride solution/methanol=1:2)

C₂₆H₂₆BrN₅O₂×HCl (520.36/556.89) Mass spectrum: (M+H)⁺=520/22 (Br) (M+Cl)⁻=554/56/58 (Cl,Br)

### (2) 2-(3-carbamimidoyl-phenyl)-N-[3-bromo-4-(pyrrolidin-1-yl-carbonyl)-phenyl]-3-[4-(ethoxycarbonyl)-phenyl]-propionamide-hydrochloride

Yield: 93% of theoretical,

R_{f} value: 0.3 (silica gel; methylene chloride/methanol=4:1+1% ammonia)

C₃₀H₃₁BrN₄O₄×HCl (591.51/627.97) Mass spectrum: (M+H)⁺=591/93 (Br) (M+Cl)⁻=625/27 (Cl, Br)

### EXAMPLE 17

Dry ampoule containing 75 mg of active substance per 10 ml

Composition

| | | |
|---|---|---|
| Active substance | 75.0 | mg |
| Mannitol | 50.0 | mg |
| water for injections | ad 10.0 | ml |

Preparation

Active substance and mannitol are dissolved in water. After packaging the solution is freeze-dried. To produce the solution ready for use, the product is dissolved in water for injections.

### EXAMPLE 18

Dry ampoule containing 35 mg of active substance per 2 ml

Composition

| | | |
|---|---|---|
| Active substance | 35.0 | mg |
| Mannitol | 100.0 | mg |
| water for injections | ad 2.0 | ml |

Preparation

Active substance and mannitol are dissolved in water. After packaging, the solution is freeze-dried.

To produce the solution ready for use, the product is dissolved in water for injections.

### EXAMPLE 19

Tablet containing 50 mg of active substance

Composition

| | |
|---|---|
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

Preparation

(1), (2) and (3) are mixed together and granulated with an aqueous solution of (4). (5) is added to the dried granulated material. From this mixture tablets are pressed, biplanar, faceted on both sides and with a dividing notch on one side. Diameter of the tablets: 9 mm.

### EXAMPLE 20

Tablet containing 350 mg of active substance

Composition

| | |
|---|---|
| (1) Active substance | 350.0 mg |
| (2) Lactose | 136.0 mg |
| (3) Maize starch | 80.0 mg |
| (4) Polyvinylpyrrolidone | 30.0 mg |
| (5) Magnesium stearate | 4.0 mg |
| | 600.0 mg |

Preparation

(1), (2) and (3) are mixed together and granulated with an aqueous solution of (4). (5) is added to the dried granulated material. From this mixture tablets are pressed, biplanar, faceted on both sides and with a dividing notch on one side. Diameter of the tablets: 12 mm.

### EXAMPLE 21

Capsules containing 50 mg of active substance

Composition

| | |
|---|---|
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

Preparation

(1) is triturated with (3). This trituration is added to the mixture of (2) and (4) with vigorous mixing.

This powder mixture is packed into size 3 hard gelatine capsules in a capsule filling machine.

### EXAMPLE 22

Capsules containing 350 mg of active substance

Composition

| | |
|---|---|
| (1) Active substance | 350.0 mg |
| (2) Dried maize starch | 46.0 mg |
| (3) Powdered lactose | 30.0 mg |
| (4) Magnesium stearate | 4.0 mg |
| | 430.0 mg |

Preparation

(1) is triturated with (3). This trituration is added to the mixture of (2) and (4) with vigorous mixing.

This powder mixture is packed into size 0 hard gelatine capsules in a capsule filling machine.

### EXAMPLE 23

Suppositories containing 100 mg of active substance

1 suppository contains:

| | |
|---|---|
| Active substance | 100.0 mg |
| Polyethyleneglycol (M.W. 1500) | 600.0 mg |
| Polyethyleneglycol (M.W. 6000) | 460.0 mg |
| Polyethylenesorbitan monostearate | 840.0 mg |
| | 2,000.0 mg |

Preparation

The polyethyleneglycol is melted together with polyethylenesorbitan monostearate. At 40° C. the ground active substance is homogeneously dispersed in the melt. This is then cooled to 38° C. and poured into slightly chilled suppository moulds.

What is claimed is:

1. A carboxylic acid amide of the formula 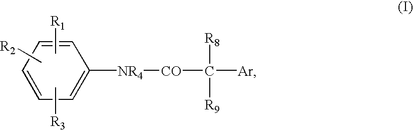
wherein:
R₁ denotes a C₃₋₇-cycloalkyl-carbonyl group wherein
the methylene group in the 3 or 4 position in a C₅₋₇-cycloalkyl-carbonyl group may be replaced by an —NH group wherein
the hydrogen atom of the —NH group may be replaced by a C₁₋₃-alkyl, C₁₋₃-alkyl-carbonyl, phenylcarbonyl or phenylsulphonyl group,
a C₁₋₆-alkylcarbonyl group optionally terminally substituted in the alkyl moiety by an amino, C₁₋₃-alkylamino or di-(C₁₋₃-alkyl)-amino group,
a group of formula
R_{f}R_{g}N—(CH₂)ₘ(Rₕ)N—CO
wherein
R_{f}, R_{g} and Rₕ independently of one another each denote a hydrogen atom or a
C₁₋₃-alkyl group and
m denotes one of the numbers 2, 3, 4, 5 or 6,
a phenylcarbonyl, naphthylcarbonyl or heteroarylcarbonyl group,
a C₁₋₃-alkyl group monosubstituted by a hydroxy group or terminally disubstituted by a phenyl and a hydroxy group wherein
the phenyl substituent may be substituted by an amidino group optionally substituted by one or two C₁₋₃-alkyl groups, by a fluorine, chlorine or bromine atom, by a trifluoromethyl, C₁₋₃-alkyl or C₁₋₃-alkoxy group,
a 4- to 7-membered cycloalkyleneimino-carbonyl or cycloalkyleneimino-sulphonyl group substituted by an amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl or di-(C₁₋₃-alkyl)-aminocarbonyl group,
a C₃₋₇-cycloalkylamino group which is substituted at the nitrogen atom by a C₁₋₃-alkyl-amino-C₁₋₃-alkyl or di-(C₁₋₃-alkyl)amino-C₁₋₃-alkyl group,
or, if R₂ denotes a trifluoromethyl group and/or R₅ denotes an amino-C₁₋₃-alkyl, C₁₋₃-alkylamino-C₁₋₃-alkyl or di-(C₁₋₃-alkyl)amino-C₁₋₃-alkyl group and/or R₆ denotes a carboxy-C₁₋₃-alkoxy or C₁₋₄-alkoxy-carbonyl-C₁₋₃-alkoxy group and/or at least one of the groups R₈ or R₉ assumes a meaning other than the hydrogen atom, an unsubstituted 4- to 7-membered cycloalkyleneimino-carbonyl or cycloalkyleneimino-sulphonyl group, a C₃₋₇-cycloalkylamino or N-(C₁₋₃-alkyl)-C₃₋₇-cycloalkylamino group,
R₂ denotes a hydrogen, fluorine, chlorine or bromine atom, a C₁₋₃-alkyl group wherein the hydrogen atoms may be wholly or partly replaced by fluorine atoms, a hydroxy or C₁₋₃-alkoxy group,
R₃ denotes a hydrogen atom or a C₁₋₃-alkyl group,
R₄ denotes a hydrogen atom or a C₁₋₃-alkyl group optionally substituted by a carboxy group or a group which may be converted into a carboxy group in vivo,
Ar denotes a phenyl or naphthyl group substituted by the groups R₅, R₆ and R₇, while
R₅ denotes a cyano group, an amidino group optionally substituted by one or two C₁₋₃-alkyl groups, an amino-C₁₋₃-alkyl, C₁₋₃-alkylamino-C₁₋₃-alkyl or di-(C₁₋₃-alkyl)amino-C₁₋₃-alkyl group,
R₆ denotes a hydrogen, fluorine, chlorine or bromine atom, a trifluoromethyl, C₁₋₃-alkyl, hydroxy, hydroxy-C₁₋₃-alkyl, C₁₋₃-alkoxy, C₁₋₃-alkoxy-C₁₋₃-alkyl, carboxy, carboxy-C₁₋₃-alkyl, carboxy-C₁₋₃-alkoxy, C₁₋₄-alkoxy-carbonyl-C₁₋₃-alkoxy, phenyl-C₁₋₃-alkoxy, amino, C₁₋₃-alkylamino or di-(C₁₋₃-alkyl)amino group and
R₇ denotes a hydrogen, fluorine, chlorine or bromine atom or a C₁₋₃-alkyl group,
or a thienylene, thiazolylene, pyridinylene, pyrimidinylene, pyrazinylene or pyridazinylene group optionally substituted in the carbon skeleton by a C₁₋₃-alkyl group,
R₈ and R₉, which may be identical or different, each denote a hydrogen atom, a C₁₋₃-alkyl group optionally substituted by a phenyl or heteroaryl group or an amino group optionally substituted by one or two C₁₋₃-alkyl or C₁₋₃-alkyl-carbonyl groups, while the term heteroaryl group mentioned above denotes a 5-membered heteroaryl group bound via a carbon or nitrogen atom which contains
an imino group optionally substituted by a C₁₋₄-alkyl or C₁₋₄-alkyl-carbonyl group, an oxygen or sulphur atom,
an imino group optionally substituted by a C₁₋₄-alkyl group or an oxygen or sulphur atom and additionally a nitrogen atom,
an imino group optionally substituted by a C₁₋₄-alkyl group and two nitrogen atoms or
an oxygen or sulphur atom and two nitrogen atoms, or a 6-membered heteroaryl group which contains one or two nitrogen atoms,
while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety, and the unsubstituted or monosubstituted phenyl and naphthyl groups mentioned in the definition of the abovementioned groups, or the unsubstituted or monosubstituted phenyl and naphthyl groups contained in these groups, as well as the abovementioned heteroaryl groups may additionally be substituted at a carbon atom in each case by a fluorine, chlorine or bromine atom, by a trifluoromethyl, C₁₋₃-alkyl, C₁₋₃-alkoxy or C₁₋₃-alkoxy-carbonyl group, unless otherwise stated,
the carboxy groups mentioned in the definition of the abovementioned groups may be replaced by a group which may be converted in vivo into a carboxy group or by a group which is negatively charged under physiological conditions, and
the amino and imino groups mentioned in the definition of the abovementioned groups may be substituted by a group which can be cleaved in vivo,
and the compounds
2-(5-carbamimidoyl-2-hydroxy-phenyl)-N-[3-chloro-4-(pyrrolidin-1-yl-carbonyl)-phenyl]-acetamide,
2-(5-carbamimidoyl-2-hydroxy-phenyl)-N-[3-methyl-4-(pyrrolidin-1-yl-sulphonyl)-phenyl]-acetamide,
2-(5-carbamimidoyl-2-hydroxy-phenyl)-N-[3-bromo-4-(pyrrolidin-1-yl-carbonyl)-phenyl]-acetamide and
2-(5-carbamidoyl-2-hydroxy-phenyl)-N-[3-methoxy-4-(pyrrolidin-1-yl-carbonyl)-phenyl]-acetamide,
or a salt thereof.

2. A carboxylic acid amide of the formula I according to claim 1, wherein:
R₁ denotes a C₅₋₇-cycloalkyl-carbonyl group wherein the methylene group in the 3 or 4 position is replaced by an —NH group wherein
the hydrogen atom may be replaced by a C₁₋₃-alkyl, C₁₋₃-alkyl-carbonyl or phenyl-carbonyl group,
a C₁₋₃-alkyl-carbonyl group optionally terminally substituted in the alkyl moiety by a C₁₋₃-alkylamino or di-(C₁₋₃-alkyl)-amino group,
a group of formula
R_{f}R_{g}N—(CH₂)ₘ—(Rₕ)N—CO,
wherein
R_{f}, R_{g} and Rₕ independently of one another each denote a hydrogen atom or a C₁₋₃-alkyl group and
m denotes one of the numbers 2, 3 or 4,
a phenylcarbonyl or heteroarylcarbonyl group,
while the heteroaryl moiety contains a 6-membered heteroaryl group which contains one or two nitrogen atoms and to which a phenyl ring may be fused via two adjacent carbon atoms, while the bicyclic heteroaryl groups thus formed may be bound via the heteroaromatic or carbocyclic moiety, e.g. a 2-pyridyl, 3-pyridyl, 4-pyridyl, pyrazinyl, pyrimidinyl, pyridazinyl, quinolinyl, isoquinolinyl, quinoxalinyl or quinazolinyl group,
a C₁₋₃-alkyl group monosubstituted by a hydroxy group or terminally disubstituted by a phenyl group and a hydroxy group wherein p2 the phenyl substituent may be substituted by an amidino group optionally substituted by one or two C₁₋₃-alkyl groups, by a fluorine, chlorine or bromine atom, by a trifluoromethyl, C₁₋₃-alkyl or C₁₋₃-alkoxy group,
a 4- to 7-membered cycloalkyleneimino-carbonyl group substituted by an amino-C₁₋₃-alkyl, C₁₋₃-alkylamino-C₁₋₃-alkyl, di-(C₁₋₃-alkyl)-amino-C₁₋₃-alkyl, C₁₋₄-alkoxy-carbonyl-amino-C₁₋₃-alkyl, aminocarbonyl, Co ₃-alkylamino-carbonyl or di-(C₁₋₃-alkyl)-aminocarbonyl group,
a C₅₋₇-cycloalkylamino group which is substituted at the nitrogen atom by a C₁₋₃-alkyl-amino-C₁₋₃-alkyl or di-(C₁₋₃-alkyl)amino-C₁₋₃-alkyl group,
or, if R₂ denotes a trifluoromethyl group and/or R₅ denotes an amino-C₁₋₃-alkyl or C₁₋₃-alkylamino-C₁₋₃-alkyl group and/or R₆ denotes a carboxy-C₁₋₃-alkoxy or C₁₋₄-alkoxy-carbonyl-C₁₋₃-alkoxy group and/or at least one of the groups R₈ or R₉ assumes a meaning other than the hydrogen atom, an unsubstituted 4- to 7-membered cycloalkyleneiminocarbonyl group, a C₅₋₇-cycloalkylamino or N-(C₁₋₃-alkyl)-C₅₋₇-cycloalkylamino group,
R₂ denotes a hydrogen, fluorine, chlorine or bromine atom, a C₁₋₃-alkyl, trifluoromethyl or C₁₋₃-alkoxy group,
R₃ denotes a hydrogen atom or a C₁₋₃-alkyl group,
R₄ denotes a hydrogen atom or a C₁₋₃-alkyl group,
Ar denotes a phenyl group substituted by the groups R₅ and R₆ wherein
R₅ denotes a cyano group, an amidino group optionally substituted by one or two C₁₋₃-alkyl groups, an amino-C₁₋₃-alkyl or C₁₋₃-alkylamino-C₁₋₃-alkyl group and
R₆ denotes a hydrogen, fluorine, chlorine or bromine atom, a trifluoromethyl, C₁₋₃-alkyl, hydroxy, C₁₋₃-alkoxy, carboxy-C₁₋₃-alkoxy or C₁₋₄-alkoxy-carbonyl-C₁₋₃-alkoxy group, and
R₈ and R₉, which may be identical or different, each denote a hydrogen atom, a C₁₋₃-alkyl group optionally substituted by an phenyl or pyridinyl group or an amino group optionally substituted by one or two C₁₋₃-alkyl or C₁₋₃-alkyl-carbonyl groups,
while the unsubstituted or monosubstituted phenyl groups mentioned in the definition of the abovementioned groups, or the unsubstituted or monosubstituted phenyl moieties contained in these groups, as well as the abovementioned heteroaryl groups may additionally be substituted at a carbon atom in each case by a fluorine, chlorine or bromine atom, by a trifluoromethyl, C₁₋₃-alkyl, C₁₋₃-alkoxy or C₁₋₃-alkoxy-carbonyl group, unless otherwise stated,
and the compounds
2-(5-carbamimidoyl-2-hydroxy-phenyl)-N-[3-chloro-4-(pyrrolidin-1-yl-carbonyl)-phenyl]-acetamide,
2-(5-carbamimidoyl-2-hydroxy-phenyl)-N-[3-methyl-4-(pyrrolidin-1-yl-sulphonyl)-phenyl]-acetamide,
2-(5-carbamimidoyl-2-hydroxy-phenyl)-N-[3-bromo-4-(pyrrolidin-1-yl-carbonyl)-phenyl]-acetamide and
2-(5-carbamidoyl-2-hydroxy-phenyl)-N-[3-methoxy-4-(pyrrolidin-1-yl-carbonyl)-phenyl]-acetamide,
or a salt thereof.

3. A carboxylic acid amide of the formula I according to claim 1, wherein:
the groups R₁ to R₄, R₈ and R₉ are defined as in claim 1 or 2, but R₁ in the 4 position is bound to the phenyl group contained in formula I and
Ar denotes a phenyl group disubstituted by the groups R₅ and R_{6,} while
R₅ is bound in the 3 position if R₆ denotes a hydrogen atom, or is bound in the 5 position if R₆ assumes a meaning other than the hydrogen atom, and an amidino group optionally substituted by one or two C₁₋₃-alkyl groups, an amino-C₁₋₃-alkyl or C₁₋₃-alkylamino-C₁₋₃-alkyl group and
R₆ denotes a hydrogen atom or a trifluoromethyl, C₁₋₃-alkyl, hydroxy, C₁₋₃-alkoxy, carboxy-C₁₋₃-alkoxy or C₁₋₄-alkoxy-carbonyl-C₁₋₃-alkoxy group bound in the 2 position,
and the compounds
2-(5-carbamimidoyl-2-hydroxy-phenyl)-N-[3-chloro-4-(pyrrolidin-1-yl-carbonyl)-phenyl]-acetamide and
2-(5-carbamimidoyl-2-hydroxy-phenyl)-N-[3-bromo-4-(pyrrolidin-1-yl-carbonyl)-phenyl]-acetamide,
or a salt thereof.

4. A carboxylic acid amide of the formula I according to claim 1, wherein:
R₁ is bound in the 4 position of the phenyl group of formula I and denotes
a C₅₋₇-cycloalkyl-carbonyl group wherein the methylene group in the 3 or 4 position is replaced by an —NH group,
a phenylcarbonyl or pyridylcarbonyl group optionally substituted by a fluorine, chlorine or bromine atom or by a C₁₋₃-alkyl group,
a C₁₋₃-alkyl group terminally disubstituted by a phenyl and a hydroxy group wherein
the phenyl substituent may be monosubstituted by a C₁₋₃-alkyl or an amidino group or may be disubstituted by a C₁₋₃-alkyl and an amidino group,
a 5- to 7-membered cycloalkyleneimino-carbonyl group substituted by an amino-C₁₋₃-alkyl, C₁₋₃-alkylamino-C₁₋₃-alkyl, C₁₋₄-alkoxy-carbonyl-amino-C₁₋₃-alkyl, aminocarbonyl or C₁₋₃-alkylamino-carbonyl group,
or, if R₂ denotes a trifluoromethyl group and/or R₅ denotes an amino-C₁₋₃-alkyl group and/or R₆ denotes a carboxy-C₁₋₃-alkoxy or C₁₋₄-alkoxy-carbonyl-C₁₋₃-alkoxy group and/or at least one of the groups R₈ or R₉ assumes a meaning other than the hydrogen atom, an unsubstituted 5- to 7-membered cycloalkyleneimino-carbonyl or cycloalkyleneimino-sulphonyl group and
R₂ denotes a hydrogen atom or a substitutent bound in the 3 position of the phenyl group, selected from among fluorine, chlorine, bromine, C₁₋₃-alkyl, C₁₋₃-alkoxy and trifluoromethyl,
R₃ and R₄ each denote a hydrogen atom,
Ar denotes a phenyl group substituted by the groups R₅ and R₆ wherein
R₅ is bound in the 3 position if R₆ denotes a hydrogen atom, or is bound in the 5 position if R₆ assumes a meaning other than the hydrogen atom, and an amidino or amino-C₁₋₃-alkyl group and
R₆ denotes a hydrogen atom or a hydroxy, C₁₋₃-alkoxy, carboxy-C₁₋₃-alkoxy or C₁₋₄-alkoxy-carbonyl-C₁₋₃-alkoxy group bound in the 2 position, and
R₈ and R₉, which may be identical or different, each denote a hydrogen atom, a C₁₋₃-alkyl group optionally substituted by a phenyl, 4-(C₁₋₃-alkoxy-carbonyl)-phenyl or pyridinyl group or an amino group optionally substituted by one or two C₁₋₃-alkyl or C₁₋₃-alkyl-carbonyl groups,
and the compounds
2-(5-carbamimidoyl-2-hydroxy-phenyl)-N-[3-chloro-4-(pyrrolidin-1-yl-carbonyl)-phenyl]-acetamide and
2-(5-carbamimidoyl-2-hydroxy-phenyl)-N-[3-bromo-4-(pyrrolidin-1-yl-carbonyl)-phenyl]-acetamide,
or a salt thereof.

5. A compound selected from the group consisting of:
(1) (L)-2-(5-carbamimidoyl-2-hydroxy-phenyl)-N-[3-methyl-4-(2-aminocarbonyl-pyrrolidin-1-yl-carbonyl)-phenyl]-acetamide,
(2) 2-(5-carbamimidoyl-2-hydroxy-phenyl)-N-{3-methyl-4-[2-(tert.butoxycarbonyl-aminomethyl)-piperidin-1-yl-carbonyl]-phenyl}-acetamide,
(3) 2-(5-aminomethyl-2-hydroxy-phenyl)-N-[3-methyl-4-(pyrrolidin-1-yl-carbonyl)-phenyl]-acetamide,
(4) 2-(3-carbamimidoyl-phenyl)-N-[3-methyl-4-(pyrrolidin-1-yl-carbonyl)-phenyl]-isobutyramide,
(5) 2-(5-carbamimidoyl-2-ethoxycarbonylmethyloxy-phenyl)-N-[3-methyl-4-(pyrrolidin-1-yl-carbonyl)-phenyl]-acetamide,
(6) 2-(5-carbamimidoyl-2-carboxymethyloxy-phenyl)-N-[3-methyl-4-(pyrrolidin-1-yl-carbonyl)-phenyl]-acetamide,
(7) 2-(5-carbamimidoyl-2-hydroxy-phenyl)-N-[3-methyl-4-(piperidin-3-yl-carbonyl)-phenyl]-acetamide,
(8) 2-(5-carbamimidoyl-2-hydroxy-phenyl)-N-(3-methyl-4-benzoyl-phenyl)-acetamide,
(9) 2-(5-carbamimidoyl-2-hydroxy-phenyl)-N-[3-methyl-4-(1-hydroxy-1-phenyl-methyl)-phenyl]-acetamide,
(10) 2-(5-carbamimidoyl-2-hydroxy-phenyl)-N-{4-[1-(3-carbamimidoyl-phenyl)-1-hydroxy-methyl]-3-methyl-phenyl}-acetamide,
(11) 2-(3-carbamidoyl-phenyl)-N-[3-methyl-4-(pyridin-3-yl-carbonyl)-phenyl]-isobutyramide,
(12) 2-(5-carbamimidoyl-2-hydroxy-phenyl)-N-[3-methyl-4-(pyrrolidin-1-yl-carbonyl)-phenyl]-isobutyramide,
(13) 2-(5-carbamimidoyl-2-hydroxy-phenyl)-N-[3-chloro-4-(pyrrolidin-1-yl-carbonyl)-phenyl]-acetamide,
(14) 2-(3-carbamimidoyl-phenyl)-N-[3-methyl-4-(pyrrolidin-1-yl-carbonyl)-phenyl]-2-amino-acetamide,
(15) 2-(3-carbamimidoyl-phenyl)-N-[3-methyl-4-(pyrrolidin-1-yl-carbonyl)-phenyl]-2-(acetylamino)-acetamide,
(16) 2-(5-carbamimidoyl-2-hydroxy-phenyl)-N-[3-methyl-4-(pyrrolidin-1-yl-sulphonyl)-phenyl]-acetamide,
(17) 2-(5-carbamimidoyl-2-hydroxy-phenyl)-N-[3-methyl-4-(pyrrolidin-1-yl-carbonyl)--phenyl]-propionamide,
(18) 2-(5-carbamimidoyl-2-hydroxy-phenyl)-N-[3-trifluoromethyl-4-(pyrrolidin-1-yl-carbonyl)-phenyl]-acetamide,
(19) 2-(5-carbamimidoyl-2-hydroxy-phenyl)-N-[4-(pyrrolidin-1-yl-carbonyl)-3-trifluoromethyl-phenyl]-propionamide,
(20) 2-(5-carbamimidoyl-2-hydroxy-phenyl)-N-[3-bromo-4-(pyrrolidin-1-yl-carbonyl)-phenyl]-acetamide,
(21) 2-(5-carbamimidoyl-2-hydroxy-phenyl)-N-[3-bromo-4-(pyrrolidin-1-yl-carbonyl)-phenyl]-propionamide,
(22) 2-(5-carbamidoyl-2-hydroxy-phenyl)-N-[3-methoxy-4-(pyrrolidin-1-yl-carbonyl)-phenyl]-acetamide,
(23) 2-(3-carbamimidoyl-phenyl)-N-[3-bromo-4-(pyrrolidin-1-yl-carbonyl)-phenyl]-3-phenyl-propionamide,
(24) 2-(3-carbamimidoyl-phenyl)-N-[3-bromo-4-(pyrrolidin-1-yl-carbonyl)-phenyl]-3-(pyridin-4-yl)-propionamide and
(25) 2-(3-carbamimidoyl-phenyl)-N-[3-bromo-4-(pyrrolidin-1-yl-carbonyl)-phenyl]-3-[4-5 (ethoxycarbonyl)-phenyl]-propionamide,
or a derivative thereof wherein at least one amidino group is substituted by a C₁₋₆-alkoxy-carbonyl or phenylcarbonyl, or a salt thereof.

6. A physiologically acceptable salt of a compound according to claim 1, 2, 3, 4 or 5, with the exception of those compounds wherein Ar denotes a phenyl or naphthyl group substituted by the groups R₅, R₆ and R₇, and R₅ denotes a cyano group.

7. A pharmaceutical composition comprising a compound in accordance with claim 1, 2, 3 or 4, with the exception of those compounds wherein Ar denotes a phenyl or naphthyl group substituted by the groups R₅, R₆ and R₇ and R₅ denotes a cyano group, or a physiologically acceptable salt thereof, together with one or more inert carriers and/or diluents.

8. A method for treating thrombus formation which method comprises administering to a host in need of such treatment an antithrombotic amount of a compound in accordance with claim 1, 2, 3 or 4, with the exception of those compounds wherein Ar denotes a phenyl or naphthyl group substituted by the groups R₅, R₆ and R₇ and R₅ denotes a cyano group, or a physiologically acceptable salt thereof.
